(19) United States
(12) Patent Application Publication
(10) Pub. No.: US 2012/0028887 A1
(43) Pub. Date: Feb. 2, 2012

(54) LIPOPEPTIDE INHIBITORS OF HIV-1

(75) Inventors: Yechiel Shai, Yehud (IL); Yael Wexler-Cohen, Rehovot (IL)

(73) Assignee: YEDA RESEARCH AND DEVELOPMENT CO. LTD., Rehovot (IL)

(21) Appl. No.: 13/260,902

(22) PCT Filed: Mar. 28, 2010
(86) PCT No.: PCT/IL10/00271

§ 371 Date: Sep. 28, 2011

Related U.S. Application Data
(60) Provisional application No. 61/165,536, filed on Apr. 1, 2009.

(51) Int. Cl. A61K 38/16; A61P 31/18; A61P 31/14; C07K 14/00; C12N 7/06

(52) U.S. Cl. 514/3.7; 530/326; 530/300; 530/325; 530/324; 435/238

(57) ABSTRACT

The invention provides lipophilic conjugates comprising a short isolated peptide coupled to a hydrophobic moiety, the peptide comprising a sequence derived from the HIV-1 gp41 N-terminal heptad repeat domain, said peptide after conjugation to the hydrophobic moiety possesses anti-fusogenic activity higher than prior to conjugation. The lipophilic conjugates are suitable for treatment of infections caused by human and non-human retroviruses, especially HIV.

### FIELD OF THE INVENTION

The present invention relates to lipophilic conjugates comprising a hydrophobic moiety coupled to peptides derived from the HIV-1 gp41 N-terminal heptad repeat domain, to pharmaceutical compositions comprising same, and use thereof as inhibitors of human and non-human retroviral, especially HIV, transmission to uninfected cells.

### BACKGROUND OF THE INVENTION

HIV-1, like other enveloped viruses utilizes a protein embedded in its membrane, termed envelope protein, to facilitate the fusion process. The envelope protein is composed of two non-covalently associated subunits; gp120 and gp41 which are organized as trimers. Gp120 is responsible for the host tropism (Clapham, P. R. and McKnigh, A. 2002, J. Gen. Viral., 83; 1809-29), while gp41, the transmembrane subunit, is responsible for the actual fusion event (Chan, D. C. and Kim, P. S., 1998, Cell, 93; 681-4). The extracellular part of gp41 is composed of several functional regions including the Fusion peptide (FP), N-terminal heptad repeat (NHR) and the C-terminal heptad repeat (CHR). The ability of the virus to fuse its own membrane with that of the hosting cell is dependent on the conversion between three identified envelope conformations: The native, metastable conformation, the Pre-Hairpin conformation, and folding into the Hairpin conformation. Binding of gp120 subunit to host receptors and co-receptors causes major conformational changes that drive the transition from the native conformation into the Pre-Hairpin conformation. In this conformation the gp41 subunit is extended leading to the insertion of the FP region into the host cell membrane. The complex representing the Hairpin is designated the “six helix bundle” (SHB) or “core” structure, and it is composed of three CHR regions which pack in an anti-parallel manner into hydrophobic grooves created on a trimeric, internal, NHR coiled-coil (Weissenhorn, W. et al., 1997, Nature, 387; 426-30). In the core structure, each of the grooves on the surface of the NHR trimer has a deep cavity termed “the pocket” that interacts with three conserved hydrophobic residues of the CHR region. These interactions are crucial for maintaining the stability of the SHB suggesting this domain as an attractive target for antiviral compounds.

Folding into the Hairpin conformation is thought to be the rate limiting step for the fusion reaction and it enables inhibition of the fusion process. This was demonstrated by the capability of different C- or N-peptides derived from the CHR region (“C-peptides”) or NHR regions (“N-peptides”) of HIV gp41, respectively, to inhibit transmission of HIV to host cells both in in vitro assays and in in vivo clinical studies. These peptides have been shown to bind their endogenous counterparts thereby preventing progression into the Hairpin conformation and arresting fusion (Root M. J. et al., 2001, Science, 291; 884-8).

For example, C-peptides, as exemplified by DP178 (also known as T20, enfuvirtide, and Fuzeon®), T651 and T649, blocked infection of target cells with potencies of 0.5 ng/ml (EC50 against HIV-1_{LAI}), 5 ng/ml (IC50; HIV-1 IIIB), and 2 ng/ml (IC50; HIV-1 IIIB), respectively. It was recently demonstrated that one of the major pathways through which DP178 inhibits fusion is through assembly with gp41 within the cellular membrane, arresting the fusion process in midway (Kliger, Y., et al., 2001, J. Biol. Chem. 276: 1391-1397). Numerous other publications have disclosed DP178, fragments, analogs and homologs thereof having anti-retroviral activity, including: U.S. Pat. Nos. 5,464,933; 5,656,480; 6,093,794; 6,133,418; 6,258,782; 6,333,395; 6,348568; 6,479,055; 6,750,008; 7,122,190, 7,273,614 and 7,456,251.

In contrast to C-peptides, N-peptides exhibit inferior inhibitory activity which is usually attributed to their tendency to aggregate (Eckert, D. M. and Kim, P. S., 2001, Proc. Natl. Acad, Sci. USA, 98; 11187-92). Nevertheless two potent N-peptides inhibitors that were intensively studied are the N36 (36 amino acids) and DP107 (37 amino acids). Attempts have been made to improve the potency of these N-peptides by two main strategies: (i) stabilization of a specific coiled-coil NHR by the addition of cysteine residues, fusion of the peptide to a known coiled-coil protein (unrelated to HIV) and by introducing repeated NHR sequences or (ii) introduction of mutations in the NHR peptide itself (Bewley, C. A. et al., 2002, J. Biol. Chem. 277, 14238-45). Although some of these attempts resulted with improved fusion inhibitors (some were found to be as potent as enfuvirtide) their preparation requires complicated manipulations. Most of these improved N-peptides were long and tended to aggregate and the shorter and simpler peptides were still considered to be too long for therapeutic purposes (>30 amino acids). Additionally these N-peptides included the highly hydrophobic C-terminal segment of the N36 peptide mainly due to its known role in the formation of ‘the pocket’ during the fusion process.

Numerous attempts to improve the potency of HIV gp41 derived peptides have been described: U.S. Pat. No. 7,090,851 relates to anti-viral peptide-albumin conjugate, wherein the anti-viral peptide is derived from DP178 and DP107 and further contains a maleimide containing group through which the peptide is covalently bound to albumin.

US Patent application No. 2008/0199483 relates to peptides selected from DP178, DP107 and related peptides and analogs thereof, exhibiting anti-viral and anti-fusogenic activity modified to provide greater stability and improved half-life in vivo. The modified peptides have a reactive group such as succinimidyl or maleimido which are capable of forming covalent bonds with one or more blood components, preferably a mobile blood component.

US Patent Application No. 2008/0096809 relates to diastereomeric peptides derived from DP178 and DP107 peptides, wherein at least two amino acid residues of the diastereomeric peptide are in the D-isomer configuration, the modified peptides display increased solubility.

In another attempt to improve the biological activity of HIV-derived C-peptides, the inventors of the current invention have found that fatty acids can replace the entire C-terminal region of DP178, known to play a crucial role in the activity of the peptide. The inhibitory activity correlated with the length of the fatty acid, with the direction of fatty acid attachment (N- or C-terminus) (Wexler-Cohen Y. and Shai Y., 2007, FASEB J., 21; 3677-84). Furthermore it was found that the fatty acid increased the local concentration of the peptide on the membrane of the cells, thereby increasing its inhibitory capability.

While the prior art C- and N-peptides have been shown useful in inhibiting viral transmission to uninfected cells, each has significant shortcomings as a therapeutic. The cost of manufacturing peptides rises exponentially with their increasing length. Their potential immunogenicity increases with their length as well. Another drawback associated with synthetic peptides relates to the solubility and stability in aqueous-based pharmaceutically acceptable carriers, such as relating to the process of making an injectable solution formulation of an HIV fusion inhibitor peptide. For example, it is difficult to achieve an injectable aqueous solution containing a synthetic peptide having an amino acid sequence of DP178 in a concentration of more than 100 mg/ml without encountering problems of solubility (wherein the formulation resembles a gel, rather than a solution, or peptide precipitates out of solution over a predetermined time period) and stability (peptide being degraded over a predetermined period of time).

Thus, there is a need for an effective retroviral fusion inhibitory peptide, especially HIV fusion inhibitor peptide. The present invention addresses these needs.

### SUMMARY OF THE INVENTION

The present invention provides retroviral fusion inhibitor peptides and lipopeptides which when added in an effective amount, can interfere with the viral fusion process mediated by HIV gp41, and more preferably, interfere with the conformational changes of gp41 necessary to effect fusion, thereby inhibiting the fusion of HIV gp41 to a target cell membrane. The peptides and lipopeptides of the invention demonstrate advantageous pharmacological properties and according to some embodiments will comprise the shortest peptide possible having these advantageous properties.

The present invention provides short lipopeptides derived from the HIV gp41 N-terminal heptad repeat (NHR) domain effective as inhibitors of human and non-human retroviral, especially HIV cell fusion. The present invention further discloses for the first time hydrophobic moieties conjugated to N36 peptide and variants thereof, the conjugates having improved cell fusion inhibitory activity.

The present invention is based in part on the finding that conjugation of a hydrophobic moiety (e.g., fatty acid, a sterol, a fat soluble vitamin) to the N- or C-terminus of an otherwise weakly active or inactive short peptide derived from the HIV gp41 NHR molecule can unexpectedly endow the peptide with superior cell fusion inhibitory activity.

HIV gp41 NHR derived peptides have not been considered as potential therapeutic agents because of their reduced activity and tendency to aggregate. Surprisingly upon conjugation of a hydrophobic moiety, their inhibitory activity is improved.

According to one aspect, the present invention provides a lipophilic conjugate comprising an isolated peptide coupled to a hydrophobic moiety; the isolated peptide comprising the sequence of formula (I) (SEQ ID NO:1):

(I)
X₁-X₂-X₃-X₄-Ser-Gly-Ile-X₅-Gln-X₆-Gln-Asn-Asn-
Leu-X₇-Arg-X₈-Ile-Glu-Ala-Gln-X₉-His

wherein:
X₁ is selected from the group consisting of an arginine and a lysine amino acid residue;
X₂ is selected from the group consisting of: glutamine, asparagines, arginine, and lysine amino acid residues;
X₃ and X₄ are each independently selected from the group consisting of leucine, isoleucine, valine and metionine amino acid residues;
X₅ is selected from the group consisting of a valine, a leucine, an isoleucine, an aspartic acid and a glutamic acid amino acid residue;
X₆ is selected from the group consisting of a glutamine, an asparagine, a glutamic acid and an aspartic acid amino acid residue;
X₇ is selected from the group consisting of a threonine, a serine, a leucine, an isoleucine and a valine amino acid residue;
X₈ is selected from the group consisting of a leucine, an isoleucine, a valine and an alanine amino acid residue;
X₉ is selected from the group consisting of an isoleucine, a leucine, a valine, a glutamine and an asparagine amino acid residue;
   wherein said hydrophobic moiety is conjugated to the N- or C-terminus of said isolated peptide, and wherein said lipophilic conjugate is capable of inhibiting protein-induced membrane fusion.

According to some currently preferred embodiments, the hydrophobic moiety is conjugated to the N-terminus of the peptide comprising the sequence of formula I.

According to some embodiments, the hydrophobic moiety may be coupled to the peptide through any other free functional group along the peptide chain, for example, to the ε-amino group of lysine. According to further embodiments, more than one hydrophobic moiety may be coupled to the peptide, through the N-terminus, C-terminus or through any other functional group along the peptide chain. Each possibility represents a separate embodiment of the present invention.

According to some embodiments, the peptide comprises the sequence of Formula (I), wherein X₁ is an arginine, X₂ is a glutamine, X₃ is a leucine and X₄ is a leucine. According to some embodiments, X₅ is a valine. According to other embodiments, X₆ is a glutamine. According to yet other embodiments, X₇ is a leucine. According to yet other embodiments X₈ is an alanine. According to some embodiments, the peptide comprises the sequence of Formula (I), wherein X₅ is a valine, X₆ is a glutamine, X₇ is a leucine and X₈ is an alanine. According to some embodiments, the peptide comprises the sequence of Formula (I), wherein X₁ is an arginine, X₂ is a glutamine, X₃ is a leucine, X₄ is a leucine, X₅ is a valine, X₆ is a glutamine, X₇ is a leucine and X₈ is an alanine.

According to certain embodiments of the invention, the isolated peptide of formula (I) comprises up to 40 amino acid residues. According to some embodiments, the peptide comprises up to 36 amino acid residues. According to some embodiments, the peptide comprises up to 30 amino acid residues. According to some other embodiments, the peptide comprises up to 27 amino acid residues. According to yet other embodiments, the peptide comprises up to 25 amino acid residues. According to further embodiments, the peptide comprises 23 amino acid residues.

According to the principles of the present invention, the isolated peptide prior to conjugation of a hydrophobic moiety is either inactive or weakly active anti-fusogenic agent. Conjugation of the hydrophobic moiety endows the peptide with an anti-fusogenic activity so that the activity is significantly higher after conjugation than prior to conjugation. According to some embodiments, conjugation of a hydrophobic moiety to a peptide of the invention enhances the anti-fusogenic activity by at least 2 fold. According to some other embodiments, conjugation of a hydrophobic moiety to a peptide of the invention enhances the anti-fusogenic activity by at least 10 fold. According to some other embodiments, conjugation of a hydrophobic moiety to a peptide of the invention enhances the anti-fusogenic activity by at least 20 fold.

According to some embodiments, the hydrophobic moiety comprises an aliphatic group comprising at least 6 carbon atoms and a reactive group through which the aliphatic group may be linked to the peptide. According to some embodiments the hydrophobic moiety comprises an aliphatic group comprising at least eight carbon atoms. Non limiting examples of such reactive groups include: a carboxyl group, a carbonyl group, an amine group and thiol group, a maleimide, an imido ester, an N-hydroxysuccinimide, alkyl halide, and aryl azide. Each possibility represents a separate embodiment of the present invention. According to some currently preferred embodiments, the hydrophobic moiety is a fatty acid. According to some other embodiments, the hydrophobic moiety is a sterol. According to some embodiments, the hydrophobic moiety is cholesterol. According to yet other embodiments, the hydrophobic moiety is a fat soluble vitamin. According to further embodiments, the fat soluble vitamin is vitamin E. Each possibility represents a separate embodiment of the present invention.

According to certain exemplary embodiments, the isolated peptide has an amino acid sequence as set forth in any one of SEQ ID NOS:2-9, as follows:

| | |
|---|---|
| RQLLSGIVQQQNNLLRAIEAQQH | SEQ ID NO: 2 |
| RQLLSGIDQEQNNLTRLIEAQIH | SEQ ID NO: 3 |
| RQLLSGIVQQQNNLLRAIEAQQHL | SEQ ID NO: 4 |
| RQLLSGIDQEQNNLTRLIEAQIHE | SEQ ID NO: 5 |
| RQLLSGIVQQQNNLLRAIEAQQHLL | SEQ ID NO: 6 |
| RQLLSGIDQEQNNLTRLIEAQIHEL | SEQ ID NO: 7 |
| RQLLSGIVQQQNNLLRAIEAQQHLLQ | SEQ ID NO: 8 |
| RQLLSGIDQEQNNLTRLIEAQIHELQ | SEQ ID NO: 9 |

Each possibility represents a separate embodiment of the present invention.

According to another aspect the present invention provides a lipophilic conjugate comprising an isolated peptide coupled to a hydrophobic moiety, the isolated peptide comprising the sequence of formula (II) SEQ ID NO:10:

(II)
Ser-Gly-Ile-X₁-Gln-X₂-Gln-Asn-Asn-Leu-X₃-Asn-X₄-
Ile-Glu-Ala-Gln-X₅-His-X₆-Leu-Gln-Leu-Thr-X₇-Trp-
X₈-Ile-Lys-Gln-Leu-X₉-Ala-Arg-Ile-Leu

wherein:
X₁ is selected from the group consisting of an aspartic acid, a glutamic acid, a valine, a leucine and an isoleucine amino acid residue;
X₂ is selected from the group consisting of an aspartic acid, a glutamic acid, an asparagine and a glutamine amino acid residue;
X₃ is selected from the group consisting of a threonine, a serine, a leucine, an isoleucine and a valine amino acid residue;
X₄ is selected from the group consisting of a leucine, an isoleucine, a valine and an alanine amino acid residue;
X₅ is selected from the group consisting of a leucine, an isoleucine, a valine, a glutamine and an asparagine, amino acid residue;
X₆ is selected from the group consisting of a leucine, an isoleucine, a valine, an aspartic acid and a glutamic acid;
X₇ is selected from the group consisting of a glutamine, an asparagine, a leucine, an isoleucine and a valine amino acid residue;
X₈ is selected from the group consisting of a lysine, an arginine and a glycine amino acid residue;
X₉ is selected from the group consisting of a leucine, an isoleucine, a valine, a glutamine and an asparagine, amino acid residue;
wherein said fatty acid is conjugated to the N-terminus or C-terminus of said isolated peptide, and wherein said lipophilic conjugate is capable of inhibiting protein-induced membrane fusion.

According to one embodiment, the peptide comprises the sequence of Formula (II), wherein X₁ is selected from a valine and an aspartic acid. According to another embodiment, X₂ is selected from a glutamine and a glutamic acid. According to another embodiment, X₃ is selected from a leucine and a threonine. According to yet another embodiment X₄ is selected from an alanine and a leucine. According to another embodiment X₅ is selected from a glutamine and an isoleucine. According to another embodiment, X₆ is selected form a leucine and a glutamic acid. According to another embodiment, X₇ is selected from a valine and a glutamine. According to another embodiment, X₈ is selected from a glycine and a lysine. According to another embodiment, X₉ is selected from a glutamine and a leucine. Each possibility represents a separate embodiment of the present invention.

According to certain embodiments of the invention, the isolated peptide of formula (II) comprises up to 40 amino acid residues. According to some embodiments, the peptide comprises 36 amino acid residues. Each possibility represents a separate embodiment of the present invention.

According to certain exemplary embodiments, the isolated peptide according to formula (II) has an amino acid sequence as set forth in any one of SEQ ID NOS: 11-12 as follows:

(SEQ ID NO: 11)
SGIVQQQNNLLRAIEAQQHLLQLTVWGIKQLQARIL
(SEQ ID NO: 12)
SGIDQEQNNLTRLIEAQIHELQLTQWKIKQLLARIL

Each possibility represents a separate embodiment of the present invention.

According to certain embodiments of the invention, the isolated peptide further comprises at least one positively charged amino acid residue at the carboxy terminus of the peptide sequence, at the amino terminus of the peptide sequence or at both termini. Preferably, at least one positively charged amino acid residue is added at the carboxy terminus of the peptide sequence. According to some embodiments of the present invention, the positively charged amino acid is a lysine. According to certain exemplary embodiments, the isolated peptide has an amino acid sequence as set forth in any one of SEQ ID NOS:13-22, as follows:

SEQ ID NO: 13
RQLLSGIVQQQNNLLRAIEAQQHK
SEQ ID NO: 14
RQLLSGIDQEQNNLTRLIEAQIHK
SEQ ID NO: 15
RQLLSGIVQQQNNLLRAIEAQQHLK
SEQ ID NO: 16
RQLLSGIDQEQNNLTRLIEAQIHEK
SEQ ID NO: 17
RQLLSGIVQQQNNLLRAIEAQQHLLK
SEQ ID NO: 18
RQLLSGIDQEQNNLTRLIEAQIHELK
SEQ ID NO: 19
RQLLSGIVQQQNNLLRAIEAQQHLLQK
SEQ ID NO: 20
RQLLSGIDQEQNNLTRLIEAQIHELQK
(SEQ ID NO: 21)
SGIVQQQNNLLRAIEAQQHLLQLTVWGIKQLQARILK
(SEQ ID NO: 22)
SGIDQEQNNLTRLIEAQIHELQLTQWKIKQLLARILK

Each possibility represents a separate embodiment of the present invention.

The peptides having the amino acid sequences: SEQ ID NO: 2, SEQ ID NO: 4, SEQ ID NO: 6, SEQ ID NO: 8, SEQ ID NO: 13, SEQ ID NO: 15, SEQ ID NO: 17 and SEQ ID NO: 19 are new and are claimed as such. Each possibility represents a separate embodiment of the present invention.

According to some embodiments of the invention, the isolated peptide is selected from all L-amino acid peptides and diastereomeric peptides. According to some embodiments the peptide comprises at least 90% L-amino acids. According to other embodiments the peptide comprises at least 95% L-amino acids.

According to some embodiments of the invention, the hydrophobic moiety is a fatty acid selected from the group consisting of saturated, unsaturated, monounsaturated, and polyunsaturated fatty acids. According to some embodiments, the fatty acids consist of at least six carbon atoms. According to some embodiments, the fatty acids consist of at least eight carbon atoms. Examples of the fatty acids that may be coupled to the peptides of the invention include, but are not limited to, hexanoic acid, heptanoic acid, octanoic acid, decanoic acid (DA), undecanoic acid (UA), dodecanoic acid (DDA; lauric acid), myristic acid (MA), palmitic acid (PA), stearic acid, arachidic acid, lignoceric acid, palmitoleic acid, oleic acid, linoleic acid, linolenic acid, α-linolenic acid, arachidonic acid, trans-hexadecanoic acid, elaidic acid, lactobacillic acid, tuberculostearic acid, docosahexaenoic acid (DHA), eicosapentaenoic acid, stearidonic acid, eicosatrienoic acid, eicosatetraenoic acid, docosapentaenoic acid and cerebronic acid. According to some embodiments, the fatty acid is selected from decanoic acid, undecanoic acid, dodecanoic acid, myristic acid, and palmitic acid.

A correlation is seen between the length of the aliphatic group coupled to the peptide and the anti-fusogenic activity observed. The longer the aliphatic group (C821 C12<C16) the higher the fusogenic inhibitory activity of the lipopeptide. According to some currently preferred embodiments, the hydrophobic moiety conjugated to the peptide comprises an aliphatic group comprising at least 16 carbon atoms (C16). According to some currently preferred embodiments, the hydrophobic moiety is a hexadecanoic acid. According to some exemplary embodiments, the hexadecanoic acid conjugated to the peptide is palmitic acid.

According to some embodiments of the present invention, the hydrophobic moiety is a fat soluble vitamin. According to another embodiment, the fat-soluble vitamin is selected from the group consisting of vitamin D, vitamin E, vitamin A and vitamin K. According to an exemplary embodiment, the fat-soluble vitamin is vitamin E. According to another exemplary embodiment, the isolated peptide is set forth in SEQ IS NO:19 and the hydrophobic moiety is vitamin E. Each possibility represents a separate embodiment of the present invention.

According to some embodiments of the present invention, the hydrophobic moiety is a sterol. According to some embodiments, the sterol is selected from a zoosterol and a phytosterols. According to an exemplary embodiment, the sterol is cholesterol.

According to some other embodiments, the hydrophobic moiety may be any other hydrophobic moiety known in the art.

It is to be understood that within the scope of the present invention are peptide derivatives, analogs or salts thereof conjugated to a hydrophobic moiety according to embodiments of the present invention, wherein the derivative, analog or salt thereof displays anti-fusogenic activity when conjugated to a hydrophobic moiety.

According to another aspect, the present invention provides a pharmaceutical composition comprising as an active ingredient a lipophilic conjugate comprising an isolated peptide coupled to a hydrophobic moiety, the isolated peptide comprising the sequence of formula (I):

(I)
X₁-X₂-X₃-X₄-Ser-Gly-Ile-X₅-Gln-X₆-Gln-Asn-Asn-
Leu-X₇-Arg-X₈-Ile-Glu-Ala-Gln-X₉-His

wherein:
X₁ is selected from the group consisting of an arginine and a lysine amino acid residue;
X₂ is selected from the group consisting of arginine, lysine, glutamine and asparagine amino acid residues;
X₃ and X₄ are each independently selected from the group consisting of leucine, isoleucine, valine and metionine amino acid residues;
X₅ is selected from the group consisting of a valine, a leucine, an isoleucine, an aspartic acid and a glutamic acid amino acid residue;
X₆ is selected from the group consisting of a glutamine, an asparagine, a glutamic acid and an aspartic acid amino acid residue;
X₇ is selected from the group consisting of a threonine, a serine, a leucine, an isoleucine and a valine amino acid residue;
X₈ is selected from the group consisting of a leucine, an isoleucine, a valine and an alanine amino acid residue;
X₉ is selected from the group consisting of an isoleucine, a leucine, a valine, a glutamine and an asparagine, amino acid residue;
   wherein said hydrophobic moiety is conjugated to the N-terminus or C-terminus of said isolated peptide, and wherein said lipophilic conjugate is capable of inhibiting protein-induced membrane fusion, and a pharmaceutically acceptable carrier or diluent.

According to some currently preferred embodiments, the hydrophobic moiety is conjugated to the N-terminus of the peptide comprising the sequence of formula I.

According to some embodiments, the hydrophobic moiety may be coupled to the peptide through any other free functional group along the peptide chain, for example, to the ε-amino group of lysine. According to further embodiments, more than one hydrophobic moiety may be coupled to the peptide, through the N-terminus, C-terminus or through any other functional group along the peptide chain.

According to another aspect, the present invention provides a pharmaceutical composition comprising as an active ingredient a lipophilic conjugate comprising an isolated peptide coupled to a hydrophobic moiety, the peptide comprises the sequence of formula (II):

(II)
Ser-Gly-Ile-X₁-Gln-X₂-Gln-Asn-Asn-Leu-X₃-Arg-X₄-
Ile-Glu-Ala-Gln-X₅-His-X₆-Leu-Gln-Leu-Thr-X₇-Trp-
X₈-Ile-Lys-Gln-Leu-X₉-Ala-Arg-Ile-Leu

wherein:
X₁ is selected from the group consisting of an aspartic acid, a glutamic acid, a valine, a leucine and an isoleucine amino acid residue;
X₂ is selected from the group consisting of an aspartic acid, a glutamic acid, an asparagine and a glutamine amino acid residue;
X₃ is selected from the group consisting of a threonine, a serine, a leucine, an isoleucine and a valine amino acid residue;
X₄ is selected from the group consisting of a leucine, an isoleucine, a valine and an alanine amino acid residue;
X₅ is selected from the group consisting of a leucine, an isoleucine, a valine, a glutamine and an asparagine, amino acid residue;
X₆ is selected from the group consisting of a leucine, an isoleucine, a valine, an aspartic acid and a glutamic acid;
X₇ is selected from the group consisting of a glutamine, an asparagine, a leucine, an isoleucine and a valine amino acid residue;
X₈ is selected from the group consisting of a lysine, an arginine and a glycine amino acid residue;
X₉ is selected from the group consisting of a leucine, an isoleucine, a valine, a glutamine and an asparagine, amino acid residue;

wherein said hydrophobic moiety is conjugated to the N-terminus or C-terminus of said isolated peptide, and wherein said lipophilic conjugate is capable of inhibiting protein-induced membrane fusion, and a pharmaceutically acceptable carrier or diluent.

According to another aspect, the present invention provides a pharmaceutical composition comprising as an active ingredient a lipophilic conjugate according to embodiments of the invention for inhibiting infection of a cell by a virus. According to some embodiments, the virus is selected from HIV and simian immunodeficiency virus.

According to another aspect, the present invention provides a pharmaceutical composition comprising as an active ingredient a peptide as set forth in SEQ ID NO:19 for inhibiting infection of a cell by a virus. According to some embodiments, the virus is selected from HIV and simian immunodeficiency virus.

The pharmaceutical composition may be formulated for any route of administration including, but not limited to, intravenous, intramuscular, intraperitoneal, nasal, intralesional and topical.

According to yet another aspect, the present invention provides a method for inhibiting protein-induced membrane fusion comprising contacting the cell with an effective amount of a lipophilic conjugate of the invention, thereby inhibiting protein-induced membrane fusion. According to some embodiments, the protein inducing membrane fusion is an envelope surface glycoprotein selected from envelope surface glycoproteins of HIV and simian immunodeficiency virus. According to some embodiments, the virus is HIV. According to other embodiments, the virus is HIV-1 and the envelope surface glycoprotein of HIV is HIV-1 gp41.

According to a further aspect, the present invention provides a method for inhibiting membrane protein assembly in a cell comprising contacting the cell with an effective amount of a lipophilic conjugate of the invention, thereby inhibiting the membrane protein assembly.

According to another aspect, the present invention provides a method for inhibiting infection by a virus to a cell comprising contacting the cell with an effective amount of a lipophilic conjugate of the invention, thereby inhibiting viral infection of the cell.

According to another aspect, the present invention provides a method for inhibiting virus replication or transmission in a subject comprising administering to the subject in need thereof a therapeutically effective amount of a pharmaceutical composition of the invention, thereby inhibiting the virus replication or transmission. According to one embodiment the subject is a human subject and the virus is HIV. According to another embodiment, the subject is an animal subject and the virus is simian immunodeficiency virus.

The pharmaceutical compositions of the present invention comprise at least one lipophilic conjugate according to the present invention, and methods of the present invention involve the administration of at least one lipophilic conjugate according to the present invention.

These and other embodiments of the present invention will be better understood in relation to the figures, description, examples, and claims that follow.

### BRIEF DESCRIPTION OF THE FIGURES

FIG. 1: Representation of the bonds created between the NHR and CHR regions in the hairpin conformation of gp41. N36, DP107, DP and DP178 are known in the art peptides, N26 is one of the peptides of the present invention.

FIG. 2: Cell-cell fusion inhibition assay for the N36 peptide and its fatty acid conjugates. Fusion inhibition is induced by the peptides. The 50% fusion inhibition concentration (IC50) values of the different peptides are presented. For each peptide at least four independent experiments were performed and were included in the calculation of the standard deviation.

FIGS. 3A-D: The inhibitory capability of the peptides as determined by the cell-cell fusion assay. (A) Illustration of the inhibitory capability for each of the N-terminally conjugated peptides; (A) N36; (B) C8-N36; (C) C12-N36; and (D) C16-N36. The peptide concentration is presented on a Log scale in order to emphasize the observed phenomenon.

FIG. 4: The inhibitory oligomeric state of the N36 conjugated peptides. The Hill's coefficient parameter for the different peptides is presented. For each peptide at least four independent experiments were performed and were included in the calculation of the standard deviation.

FIG. 5: Relative concentrations of the peptides on cells; assigning NBD-labeled peptides to cells. NBDN36, C16-N36MNBD, and NBDN36M-C16 are represented by closed squares, closed triangles, and open triangles, respectively. The negative control for a non-binding peptide, NBDGCN4, is denoted by open circles whereas the positive control for a strongly binding peptide, C16-NBDGCN4, is denoted by closed circles.

FIG. 6: Utilizing CD spectroscopy to analyze the structure of the peptides, as well as their ability to create a core structure with C34, in solution and in a membrane mimetic environment. Peptides and their complexes were measured at 10 μM in 5 mM Hepes or 1% LPC (membrane mimetic environment) in ddH2O. In the left column panels, the open circles denote the peptide signal in solution, whereas the closed circles denote the peptide signal in LPC. In the middle column panels, the open triangles represent the calculated non-interacting signal for combining an N-peptide with C34, whereas the closed triangles represent the actual experimental signals, obtained following incubation of the two peptides together. In the right column panels, the same experiment was done in LPC, whereas the calculated non-interacting and the experimental signals are represented by open and closed squares, respectively.

FIGS. 7A-B: Cell-cell fusion inhibition assay for the N36 mutants and their fatty acid conjugates as determined by the cell-cell fusion assay. (A) Fusion inhibition induced by the N36 MUTe,g peptides. The IC50 values of the different peptides are presented. For each peptide at least four independent experiments were performed and were included in the calculation of the standard deviation. (B) The inhibitory oligomeric state of the peptides indicated by Hill's coefficient parameter calculated for N36 MUTe,g peptide and its N and C terminally conjugated fatty acid. For each peptide at least four independent experiments were performed and were included in the calculation of the standard deviation.

FIGS. 8A-D: Relative concentrations of the peptides on specific cell populations. In each panel the Y axis represents the percentage of labeled peptide in target cells (with receptors), whereas the X axis represents the percentage of labeled peptide in effector cells (with envelope glycoprotein). (A) C16-N36; (B) N36-C16; (C) NBDGCN4 used as a non-binding peptide control and (D) C16-NBDGCN4 used as a strongly non-specific binding peptide control. The line drawn in each panel emphasizes the expected behavior when no preference between the different populations exists. The different data points represent rising peptide concentrations.

FIG. 9: Fusion inhibition as determined by the cell-cell fusion assay (represented by IC50 values) of the peptides C8-N26M, C12-N26M, C16-N26M, C16-N25 and C16-N23.

FIG. 10: Fusion inhibition curves as determined by the cell-cell fusion assay for N26 (circles), C12-N26 (squares), and C16-N26 (triangles) peptides. Inhibition curves were fitted to a competitive model of inhibition; the fits are represented by continuous lines.

### DETAILED DESCRIPTION OF THE INVENTION

The present invention provides lipophilic conjugates or lipopeptides comprising an isolated peptide coupled to a hydrophobic moiety, the peptide corresponding to a fragment of the transmembrane protein HIV-1 gp41 N-terminal heptad repeat (NHR). The lipopeptides of the invention are capable of binding to the transmembrane protein thereby inhibiting the functional assembly of said transmembrane protein. The lipopeptides of the present invention display anti-fusogenic and anti-viral activities and are thus useful for inhibiting various biological events associated with membrane protein assembly, especially HIV transmission to uninfected cells.

The lipopeptides of the present invention are highly advantageous over all peptides having the same amino acid sequence because of their elevated inhibitory activity and increased stability. These characteristics endow the lipopeptides with higher efficacy and higher bioavailability than those peptides comprising the same amino acid sequence. Furthermore, the present invention provides lipopeptides comprising peptide sequences as short as 23 amino acids which are advantageous not only because of their cell fusion inhibitory capabilities but by their lower manufacturing costs.

The terms “lipopeptide” and “lipophilic conjugate” as used herein refer to a peptide covalently coupled to a hydrophobic moiety. The terms lipopeptide and lipophilic conjugate are used interchangeably throughout the specification and claims.

It should be understood that a peptide of the lipophilic conjugate or lipopeptide of the invention need not be identical to the amino acid sequence of a naturally occurring membrane protein so long as it includes the required sequence that allows it to bind the membrane protein and as such is able to inhibit membrane protein assembly.

The term “membrane binding” lipophilic conjugate refer to a peptide capable of interacting or binding to membranal lipids.

The terms “membrane protein assembly” or “functional assembly” of a transmembrane protein is used herein refer to complex formation or non-covalent interaction between transmembrane proteins, which lead to membrane fusion events and/or to intracellular processes initiated by the membrane protein complex formation or membrane protein interactions. The term “membrane protein” is used herein to refer to cellular membrane proteins of human or non-human cells as well as to viral envelope proteins. It should be understood that functional assembly of a protein includes homodimerization and heterodimerization, i.e., the protein may interact with an identical protein or it may interact with a different protein. Thus, functional assembly includes, but is not limited to, an interaction between two proteins adjacent to each other to form a non-covalent complex within the same cellular membrane and an interaction between different membrane proteins present in different cells. The terms “functional assembly of a membrane protein” and “membrane protein assembly” are used interchangeably. The term “transmembrane protein” refers to a membrane protein that spans the lipid bilayer of the membrane.

The present invention encompasses lipopeptide derivatives and analogs having amino acid substitutions, and/or extensions.

The term “analog” as used herein refers to peptides according to embodiments of the invention comprising altered sequences by amino acid substitutions or chemical modifications. The amino acid substitutions may be of conserved or non-conserved nature. Conserved amino acid substitutions consist of replacing one or more amino acids of an all L-amino acid or diastereomeric peptide of the invention with amino acids of similar charge, size, and/or hydrophobicity characteristics. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the non-polar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such substitutions are known as conservative substitutions. Non-conserved substitutions consist of replacing one or more amino acids of an all L-amino acid or a diastereomeric peptide with amino acids possessing dissimilar charge, size, and/or hydrophobicity characteristics, such as, for example, substitution of a glutamic acid (E) to valine (V). The amino acid substitutions may also include non-natural amino acids.

Amino acid extensions may consist of a single amino acid residue or stretches of residues. The extensions may be made at the carboxy or amino terminal end of peptides of the invention according to formula (I) and formula (II). Such extensions will generally range from 2 to 17 amino acids in length. Preferably, the peptide comprises not more than 40 amino acid residues in total. It One or more such extensions may be introduced into a peptide so long as such extensions result in a peptide, which still exhibits anti-fusogenic activity by itself or when conjugated to a hydrophobic moiety. According to some preferred embodiments, the extensions of the peptides of the invention comprise at least one positively charged amino acid at the amino terminus, at the carboxy terminus, or at both termini of the peptide. Positively charged amino acids that may be added to the peptides of the invention include, but are not limited to, lysine, arginine, histidine, or any other non-charged amino acid derivatized to yield a positively charged amino acid.

Typically, the present invention encompasses derivatives of the lipopeptides. The term “derivative” includes any chemical derivative of the peptide having one or more residues chemically derivatized by reaction of side chains or functional groups. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 5-hydroxylysine may be substituted for lysine; 3-methylhistidine may be substituted for histidine; homoserine may be substituted or serine; and ornithine may be substituted for lysine. The term “derivative” may further include chemical derivatives of the fatty acid moieties.

The present invention provides lipopeptides comprising a peptide which comprises from about 23 to 40 amino acid residues corresponding to a fragment of a transmembrane protein. According to some embodiments, the peptides comprise the amino acid sequence of a transmembrane domain of a membrane protein.

According to the invention, the lipopeptides exhibit inhibitory activity of functional assembly of a membrane protein. The inhibitory activity of functional assembly of a membrane protein includes, but is not limited to, anti-fusogenic activity and anti-viral activity.

The terms “anti-fusogenic” and “anti-membrane fusion” and “cell fusion inhibitor”, as used herein, refer to an agent's ability to inhibit or reduce the level of membrane fusion events between two or more moieties relative to the level of membrane fusion which occurs between these moieties in the absence of the lipopeptide of the invention. The moieties may be, for example, cell membranes or viral structures, such as viral envelopes or pili. The term “anti-viral”, as used herein, refers to the compound's ability to inhibit viral infection of cells, via, for example, cell-cell fusion or free virus infection. Such infection may involve membrane fusion, as occurs in the case of enveloped viruses, or some other fusion event involving a viral structure and a cellular structure (e.g., such as the fusion of a viral pilus and bacterial membrane during bacterial conjugation). A lipopeptide of the invention exhibits an anti-fusogenic and/or anti-viral activities if the level of membrane fusion events is lower in the presence of the lipopeptide than in its absence.

Assays for cell fusion events are well known to those of skill in the art. Cell fusion assays are generally performed in vitro. Such an assay includes culturing cells, which, in the absence of any treatment, would undergo an observable level of syncytial formation. For example, uninfected cells may be incubated in the presence of cells chronically infected with a virus that induces cell fusion. Viruses that induce cell fusion include, but are not limited to, HIV, SIV, or respiratory syncytial virus.

For the cell fusion assay, cells are incubated in the presence of a lipopeptide to be assayed. For each lipopeptide, a range of lipopeptide concentrations may be tested. This range should include a control culture wherein no lipopeptide has been added.

Standard conditions for culturing cells, well known to those of ordinary skill in the art, are used. After incubation for an appropriate period, the culture is examined microscopically for the presence of multinucleated giant cells, which are indicative of cell fusion and syncytial formation. Well-known stains, such as crystal violet stain, may be used to facilitate the visualization of syncytial formation. Alternatively or additionally, cell fusion may be detected by fluorescent dye transfer between labeled donor cells such as, for example, cells expressing HIV-1 gp120-41 and acceptor cells such as, for example, mouse fibroblasts, labeled with a different fluorescent dye. Addition of a lipopeptide of the present invention inhibits dye transfer, which is indicative of inhibition of cell fusion. Another example comprised cell-lines of human T-cells, such as Jurkat E6-1 and Jurkat HXBc2 cells. Jurkat HXBc2 cells express HIV-1 HXBc2 Rev and ENV proteins, whereas Jurkat E6-1 are normal T-cells. Each cell type is labeled with either DiI or DiD lipophilic fluorescent probes, respectively. The two cell populations are co-incubated in the presence of different concentrations of the inhibitory lipopeptides. The percentage of fused cells, with or without the peptides, is collected using flow cytometry and upgraded to a FACSCalibur cell analyzer.

Other assay to evaluate the inhibitory activity of a lipopeptide in membrane protein assembly may use the ToxR system, which is a robust method for detecting homodimerization of transmembrane domains in vivo.

Assays to test anti-viral activities of a lipopeptide may be based upon measuring an enzymatic activity of a virus as a function of viral infection. If taking HIV as an example, a reverse transcriptase (RT) assay may be utilized to test a lipopeptide ability to inhibit infection of CD-4+ cells by cell-free HIV. Such an assay may comprise culturing an appropriate concentration (i.e., TCID50) of virus and CD-4+ cells in the presence of the lipopeptide to be tested. Culture conditions well known to those in the art are used. A range of lipopeptide concentrations may be used, in addition to a control culture wherein no lipopeptide has been added. After incubation for an appropriate period of culturing, a cell-free supernatant is prepared, using standard procedures, and tested for the presence of RT activity as a measure of successful infection. The RT activity may be tested using standard techniques (see Goff, S. et al., 1981, J. Virol. 38:239-248; Willey, R. et al., 1988, J. Virol. 62:139-147). Another assay to test anti-viral activities of a lipopeptide may be based upon measuring luciferase activity in cells infected with the viruses in the presence of the lipopeptides. These assays normally comprised CD4+ and co-receptor expressing cells, such as TZM-bl Hela cells. In addition, these cells contain a reporter luciferase gene which is expressed upon induction by viral proteins in infected cells. The luminescence signal in cells is decreased when incubating the inhibitory lipopeptides with the virus-cell mixture.

Standard methods, which are well known to those of skill in the art, may be utilized for assaying non-retroviral activity. See, for example, Pringle et al. (Pringle, C. R. et al., 1985, J. Medical Virology 17:377-386) for a discussion of respiratory syncytial virus and parainfluenza virus activity assay techniques.

In vivo assays may also be utilized to test, for example, the antiviral activity of the lipopeptides of the invention. To test for anti-HIV activity, for example, the in vivo model described in Barnett et al. may be used (Barnett, S. W. et al., 1994, Science 266:642-646, the content of which is incorporated by reference as if fully set forth herein).

The anti-fusogenic capability of the lipopeptides of the invention may additionally be utilized to inhibit or treat/ameliorate symptoms caused by processes involving membrane fusion events. Such events may include, for example, virus transmission via cell-cell fusion, and sperm-egg fusion. Further, the lipopeptides of the invention may be used to inhibit free viral infection or transmission of uninfected cells wherein such viral infection involves cell-cell fusion events or involves fusion of a viral structure with a host cell membrane.

Retroviral viruses whose transmission may be inhibited by the lipopeptides of the invention include, for example, human retroviruses, particularly HIV-1 and HIV-2.

The anti-viral activity of the lipopeptides of the invention may show a pronounced type and subtype specificity, i.e., specific lipopeptides may be effective in inhibiting the activity of only specific viruses. This feature of the invention presents many advantages. One such advantage, for example, lies in the field of diagnostics, wherein one can use the antiviral specificity of the lipopeptide of the invention to ascertain the identity of a viral isolate.

The peptides of the present invention can be synthesized using methods well known in the art including chemical synthesis and recombinant DNA technology. Synthesis may be performed by solid phase peptide synthesis described by Merrifield (see J. Am. Chem. Soc., 85:2149, 1964). Alternatively, the peptides of the present invention can be synthesized using standard solution methods (see, for example, Bodanszky, M., Principles of Peptide Synthesis, Springer-Verlag, 1984). Preferably, the peptides of the invention are synthesized by solid phase peptide synthesis as exemplified herein below (Example 1).

The invention further contemplates lipophilic conjugates comprising peptides composed of all L-amino acids or diasteriomeric peptides. The term “diastereomeric peptide” as used herein refers to a peptide comprising both L-amino acid residues and D-amino acid residues. The amino acid residues are represented throughout the specification and claims by three-letter codes according to IUPAC conventions. When there is no indication, the amino acid residue occurs in L isomer configuration Amino acid residues present in D isomer configuration are indicated by “D” before the residue abbreviation.

Positively charged amino acids as used herein are selected from positively charged amino acids known in the art. Examples of positively charged amino acids are lysine, arginine, and histidine. Hydrophobic amino acids as used herein are selected from hydrophobic amino acids known in the art. Examples of hydrophobic amino acids are leucine, isoleucine, glycine, alanine, and valine. Negatively charged amino acids are selected from negatively charged amino acids known in the art including, but not limited to, glutamic acid and aspartic acid.

### Hydrophobic Moieties

The term “hydrophobic” refers to the tendency of chemical moieties with nonpolar atoms to interact with each other rather than water or other polar atoms. Materials that are “hydrophobic” are, for the most part, insoluble in water. Non limiting examples of natural products with hydrophobic properties include lipids, fatty acids, phospholipids, sphingolipids, acylglycerols, waxes, sterols, steroids, terpenes, prostaglandins, thromboxanes, leukotrienes, isoprenoids, retinoids, biotin, and hydrophobic amino acids such as tryptophan, phenylalanine, isoleucine, leucine, valine, methionine, alanine, proline, and tyrosine. A chemical moiety is also hydrophobic or has hydrophobic properties if its physical properties are determined by the presence of nonpolar atoms. The term includes lipophilic groups.

The term “lipophilic group”, in the context of being attached to a peptide, refers to a group having high hydrocarbon content thereby giving the group high affinity to lipid phases. A lipophilic group can be, for example, a relatively long chain alkyl or cycloalkyl (preferably n-alkyl) group having approximately 6 to 30 carbons. The alkyl group may terminate with a hydroxyl, primary amine or any other reactive group. To further illustrate, lipophilic molecules include naturally-occurring and synthetic aromatic and non-aromatic moieties such as fatty acids, esters and alcohols, other lipid molecules, cage structures such as adamantane, and aromatic hydrocarbons such as benzene, perylene, phenanthrene, anthracene, naphthalene, pyrene, chrysene, and naphthacene.

According to some embodiments of the present invention, the hydrophobic moiety may be coupled to the N-terminal, to the C-terminal, or to any other free functional group along the peptide chain, for example, to the 6-amino group of lysine. It should be understood that the hydrophobic moiety is covalently coupled to the peptide. The terms “coupling” and “conjugation” are used herein interchangeably and refer to the chemical reaction, which results in covalent attachment of a hydrophobic moiety to a peptide to yield a lipophilic conjugate. Coupling of a hydrophobic moiety to a peptide is performed similarly to the coupling of an amino acid to a peptide during peptide synthesis. Alternatively, the coupling of a hydrophobic moiety to a peptide may be performed by any coupling method known in the art.

According to some embodiments, the hydrophobic moiety comprises an aliphatic group and a reactive group through which the aliphatic group may be linked to the peptide. Non limiting examples of such reactive groups include: a carboxyl group, a carbonyl group, an amine group, a thiol group, a hydroxyl group, a maleimide, an imido ester, an N-hydroxysuccinimide, alkyl halide, and aryl azide.

The term “aliphatic”, “aliphatic group” or “aliphatic chain, as used herein, means a straight-chain (i.e., unbranched) or branched, substituted or unsubstituted hydrocarbon chain that is completely saturated or that contains one or more unsaturated bonds. Unless otherwise specified, aliphatic groups contain at least aliphatic carbon atoms. In some embodiments, aliphatic groups contain between 6 and 30 aliphatic carbon atoms. In other embodiments, aliphatic groups contain at least 8 aliphatic carbon atoms. In other embodiments, aliphatic groups contain at least 10 aliphatic carbon atoms. In still other embodiments, aliphatic groups contain at least 12 aliphatic carbon atoms, and in yet other embodiments aliphatic groups contain at least 16 aliphatic carbon atoms. Suitable aliphatic groups include, but are not limited to, linear or branched, substituted or unsubstituted alkyl, alkenyl, alkynyl, and heteroalkyl groups.

### Chemical Definitions:

The term “alkyl” refers to a saturated, linear or branched hydrocarbon moiety, such as —CH₃—(CH₂)₄—CH₂; —CH₂—(CH₂)₅—CH₂, CH₃—(CH₂)₆—CH₂, CH₃—(CH₂)₇—CH₂, CH₃—(CH₂)₈—CH₂, CH₃—(CH₂)₉—CH₂, CH₃—(CH₂)₁₀—CH₂, CH₃—(CH₂)₁₁—CH₂, CH₃—(CH₂)₁₂—CH₂; CH₃—(CH₂)₁₃—CH₂, CH₃—(CH₂)₁₄—CH₂.
The term “alkenyl” as used herein, denotes a divalent group derived from a straight chain or branch hydrocarbon moiety containing at least 6 carbon atoms having at least one carbon-carbon double bond.
The term “heteroalkyl” refers to an alkyl or an alkenyl moiety having at least one heteroatom (e.g., N, O, or S). Preferred are heteroalkylenes having at least one 0.
The term “unsaturated”, as used herein, means that a moiety has one or more units of unsaturation.
According to some currently preferred embodiments, the hydrophobic moiety is a fatty acid.

Fatty acids: The fatty acid that can be coupled to the peptides of the invention is selected from saturated, unsaturated, monounsaturated, and polyunsaturated fatty acids. Typically, the fatty acid consists of at least six carbon atoms, preferably, at least eight carbon atoms. According to some embodiments, the fatty acid is an essential fatty acid. “Essential fatty acids” may refer to certain fatty acids, in particular polyunsaturated fatty acids that an organism must ingest in order to survive, being unable to synthesize the particular essential fatty acid de novo. Examples include the essential fatty acid C9, C12-linoleic acid and their structural variants. Essential fatty acids may be found in nature or produced synthetically. Non limiting examples to fatty acids according to some embodiments of the invention include: decanoic acid (DA), undecanoic acid (UA), dodecanoic acid (lauric acid), myristic acid (MA), palmitic acid (PA), stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, docosahexaenoic acid (DHA), eicosapentaenoic acid, stearidonic acid, eicosatrienoic acid, eicosatetraenoic acid, docosapentaenoic acid and cerebronic acid. conjugated linolenic acid, omega 3 fatty acids (for example: docosahexaenoic acid (DHA), eicosapentaenoic acid, α-linolenic acid, stearidonic acid eicosatrienoic acid, eicosatetraenoic acid, docosapentaenoic acid and glycerol ester derivatives thereof), omega 6 fatty acids (for example: linoleic acid, gamma-linolenic acid, eicosadienoic acid, dihomo-gamma-linolenic acid, arachidonic acid, docosadienoic acid, adrenic acid, docosapentaenoic acid and calendic acid), omega 9 fatty acids (for example: oleic acid, eicosenoic acid, mead acid, erucic acid and nervonic acid), polyunsaturated fatty acids, long-chained polyunsaturated fatty acids, arachidonic acid, monounsaturated fatty acids, precursors of fatty acids, and derivatives of fatty acids.

It should be emphasized that any fatty acid having at least six carbon atoms could be coupled to the peptides of the invention so long as the anti-fusogenic activity of the conjugate is enhanced.

Vitamins: In certain embodiments, the present invention relates to vitamins selected from the group consisting of: vitamin A, vitamin D, vitamin E and vitamin K.

According to other embodiments, the present invention relates to any other vitamin, salts and derivatives thereof known in the art. According to other embodiments, the vitamins can be from any source known in the art. According to certain embodiments the vitamin D is selected from the group consisting of vitamin D2 (ergocalciferol), vitamin D3 (cholecalciferol) and any other vitamin D or its derivatives known in the art.

According to other embodiments, the present invention relates to vitamin D salts and derivatives thereof. According to other embodiments, the vitamin E is selected from the group consisting of α, β, γ, δ-tocopherols and α, β, γ, δ-tocotrienol and any other vitamin E known in the art. According to other embodiments, the present invention relates to vitamin E salts (e.g., vitamin E phosphate) and derivatives (e.g., tocopheryl sorbate, tocopheryl acetate, tocopheryl succinate, and other tocopheryl esters). According to additional embodiments, the vitamin A is selected from the group consisting of retinol, retinal, retinoic acid and any other vitamin A known in the art. According to other embodiments, the present invention relates to vitamin A salts and derivatives thereof. According to other embodiments, the vitamin K is selected from the group consisting of vitamin K1 (phytonadione), vitamin K2 (menaquinone), vitamin K3 (menadione), vitamin K4, vitamin K5, vitamin K6, vitamin K7, and their salts and derivatives.

Sterols: According to one embodiment refers to a steroid with a hydroxyl group at the 3-position of the A-ring. According to another embodiment, the term refers to a steroid having the following structure:

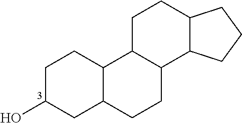

In another embodiment, the sterol is a zoosterol. In yet another embodiment, the sterol is a phytosterols. According to one embodiment, the zoosterol is cholesterol or derivatives thereof. Non limiting examples of phytosterols include stigmasterol, beta-sitosterol, campesterol, ergosterol (provitamin D2), brassicasterol, delta-7-stigmasterol and delta-7-avenasterol.

Without wishing to be bound to any mechanism of action, it is appreciated that coupling of a hydrophobic moiety to a peptide is aimed at increasing peptide hydrophobicity, optionally its oligomerization in solution, and thus endowing it with anti-fusogenic activity.

### Pharmaceutical Composition

The present invention provides pharmaceutical compositions comprising the lipophilic conjugates of the invention and a cosmetically and/or pharmaceutically acceptable carrier. The term “pharmaceutically acceptable carrier” refers to a vehicle which delivers the active components to the intended target and which does not cause harm to humans or other recipient organisms. As used herein, “pharmaceutical” will be understood to encompass both human and animal pharmaceuticals. Useful carriers include, for example, water, acetone, ethanol, ethylene glycol, propylene glycol, butane-1,3-diol, isopropyl myristate, isopropyl palmitate, or mineral oil. Methodology and components for formulation of pharmaceutical compositions are well known, and can be found, for example, in Remington's Pharmaceutical Sciences, Eighteenth Edition, A. R. Gennaro, Ed., Mack Publishing Co. Easton Pa., 1990. The pharmaceutical composition may be formulated in any form appropriate to the mode of administration, for example, solutions, colloidal dispersions, emulsions (oil-in-water or water-in-oil), suspensions, creams, lotions, gels, foams, sprays, aerosol, ointment, tablets, suppositories, and the like.

The pharmaceutical compositions can also comprise other optional materials, which may be chosen depending on the carrier and/or the intended use of the composition. Additional components include, but are not limited to, antioxidants, chelating agents, emulsion stabilizers, e.g., carbomer, preservatives, e.g., methyl paraben, fragrances, humectants, e.g., glycerin, waterproofing agents, e.g., PVP/Eicosene Copolymer, water soluble film-formers, e.g., hydroxypropyl methylcellulose, oil-soluble film formers, cationic or anionic polymers, and the like.

The pharmaceutical compositions useful in the practice of the present invention comprise a lipopeptide of the invention optionally formulated into the pharmaceutical composition as a pharmaceutically acceptable salt form. Pharmaceutically acceptable salts include the acid addition salts (formed with the free amino groups of the polypeptide), which are formed with inorganic acids, such as for example, hydrochloric or phosphoric acid, or with organic acids such as acetic, oxalic, tartaric, and the like. Suitable bases capable of forming salts with the lipopeptides of the present invention include, but are not limited to, inorganic bases such as sodium hydroxide, ammonium hydroxide, potassium hydroxide and the like; and organic bases such as mono-, di- and tri-alkyl and aryl amines (e.g. triethylamine, diisopropyl amine, methyl amine, dimethyl amine and the like) and optionally substituted ethanolamines (e.g. ethanolamine, diethanolamine and the like).

The anti-fusogenic capability of the short lipopeptides of the invention may additionally be utilized to inhibit or treat/ameliorate symptoms caused by processes involving membrane fusion events. Such events may include, for example, virus transmission via cell-cell fusion, and sperm-egg fusion. Further, the short lipopeptides of the invention may be used to inhibit free viral infection or transmission of uninfected cells wherein such viral infection involves cell-cell fusion events or involves fusion of a viral structure with a host cell membrane.

Retroviral viruses whose transmission may be inhibited by the short lipopeptides of the invention include, for example, human retroviruses, particularly HIV, even more particularly HIV-1.

One such advantage, for example, lies in the field of diagnostics, wherein one can use the anti-fusogenic specificity of the lipopeptide of the invention to ascertain the identity of a viral isolate.

According to another aspect, the present invention provides a pharmaceutical composition comprising a therapeutically effective amount of a lipophilic conjugate according to the principles of the present invention and a pharmaceutically acceptable carrier, the lipophilic conjugate capable of inhibiting fusion of a transmembrane protein, without wishing to be bound by theory or mechanism of action, the anti-fusogenic activity of the lipopeptides of the invention originated from their ability to interfere with the functional assembly of a viral transmembrane protein.

A pharmaceutical composition useful in the practice of the present invention typically contains a lipopeptide of the invention formulated into the pharmaceutical composition as a pharmaceutically acceptable salt form. Pharmaceutically acceptable salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

Pharmaceutically acceptable salts may be prepared from pharmaceutically acceptable non-toxic bases including inorganic or organic bases. Salts derived from inorganic bases include aluminum, ammonium, calcium, copper, ferric, ferrous, lithium, magnesium, manganic, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

A therapeutically effective amount of a lipophilic conjugate of the invention is an amount that when administered to a patient is capable of exerting an inhibitory activity of functional assembly of a membrane protein and hence of membrane fusion events such as, for example, viral infection, bacterial infection, and intracellular processes involving protein membrane assembly. According to some embodiments, a pharmaceutical composition of the present invention is useful for inhibiting a viral disease in a patient as described further herein. According to such embodiments, a therapeutically effective amount is an amount that when administered to a patient is sufficient to inhibit, preferably to eradicate, a viral disease.

The pharmaceutical compositions of the present invention comprise at least one lipophilic conjugate according to the present invention, and methods of the present invention involve the administration of at least one lipophilic conjugate according to the present invention.

It is to be further understood that the lipophilic conjugates of the invention may be therapeutically used in combination with additional peptides and lipopeptides that target different sequences along the transmembrane protein. For example, a short lipopeptide of the invention derived from the N-terminus of HIV-1 gp41 NHR, can work together with a peptide derived from the HIV-1 gp41 CHR sequence targeting the pocket region. Peptides and lipopeptides comprising sequences that cannot bind each other can potentially be combined. Such sequences would not neutralize each other's effect but rather enhance it.

Since most research so far has been concentrated on targeting peptides to interfere with the formation of known pocket regions (e.g. DP178, DP107 and even N36), having short-lipopeptides that target different sequences along the HIV-1 gp41 would be advantageous.

The preparation of pharmaceutical compositions, which contain peptides as active ingredients, is well known in the art. Typically, such compositions are prepared as injectable, either as liquid solutions or suspensions. However, solid forms, which can be suspended or solubilized prior to injection, can also be prepared. The preparation can also be emulsified. The active therapeutic ingredient is mixed with inorganic and/or organic carriers, which are pharmaceutically acceptable and compatible with the active ingredient. Carriers are pharmaceutically acceptable excipients (vehicles) comprising more or less inert substances that are added to a pharmaceutical composition to confer suitable consistency or form to the composition. Suitable carriers are, for example, water, saline, dextrose, glycerol, ethanol, or the like and combinations thereof. In addition, if desired, the composition can contain minor amounts of auxiliary substances such as wetting or emulsifying agents, pH buffering agents, stabilizers, and anti-oxidants, which enhance the effectiveness of the active ingredient.

The pharmaceutical composition can be delivered by a variety of means including intravenous, intramuscularly, infusion, intranasal, intraperitoneal, subcutaneous, rectal, topical, or into other regions, such as into synovial fluids. However delivery of the composition transdermally is also contemplated, such by diffusion via a transdermal patch.

According to another aspect the present invention provides a method for inhibiting membrane protein assembly in a cell comprising contacting the cell with an effective amount of a membrane binding lipophilic conjugate according to the principles of the present invention, thereby inhibiting membrane protein assembly.

According to a further aspect, the present invention provides a method for inhibiting infection of a cell by a virus comprising contacting the cell with an effective amount of a membrane binding lipophilic conjugates according to the principles of the present invention, thereby inhibiting the infection of the cell.

According to still a further aspect, the present invention provides a method for inhibiting virus replication and transmission in a subject comprising administering to the subject a therapeutically effective amount of a pharmaceutical composition comprising a lipophilic conjugate according to the principles of the present invention dispersed in a pharmaceutically acceptable carrier or diluent.

Patients in which the inhibition of viral replication would be clinically useful include patients suffering from diseases transmitted by various viruses including, for example, human retroviruses, particularly HIV-1 and HIV-2.

The pharmaceutical composition is administered in a manner compatible with the dosage formulation, and in a therapeutically effective amount. The quantity to be administered depends on the subject to be treated, and the capacity of the subject's blood hemostatic system to utilize the active ingredient. Precise amounts of active ingredient required to be administered depend on the judgment of the practitioner and are peculiar to each individual.

Methods of treating a disease according to the invention may include administration of the pharmaceutical compositions of the present invention as a single active agent, or in combination with additional methods of treatment. The methods of treatment of the invention may be in parallel to, prior to, or following additional methods of treatment. Methods of treating a disease according to the invention may include administration of the pharmaceutical compositions of the present invention as a single active agent, or in combination with additional methods of treatment. The methods of treatment of the invention may be in parallel to, prior to, or following additional methods of treatment.

The following examples are presented in order to more fully illustrate some embodiments of the invention. They should, in no way be construed, however, as limiting the broad scope of the invention.

### EXAMPLES

Materials—F-Moc amino acids including lysine with an MTT side chain protecting group and F-Moc Rink Amide MBHA resin were purchased from Nova-biochem AG (Laufelfinger, Switzerland). Other peptide synthesis reagents, fatty acids, namely, octanoic acid (C8), dodecanoic acid (C12), and hexadecanoic acid (C16), LPC (lysophosphatidylcholine), and PBS were purchased from Sigma Chemical Co. (Israel). DiD (DiIC₁₈(5) or 1,1′-dioctadecyl-3,3,3′,3′,-tetramethylindodicarbocyanine, 4-chlorobenzenesulfonate salt), DiI (1,1′-dioctadecyl-3,3,3′,3′,0 tetramethylinocarbocyanine perchlorate) lypophilic fluorescent probes were obtained from Biotium (California, USA). Buffers were prepared in double-distilled water.
Cell Lines and Reagents—Cell culture reagents and media were purchased from Biological Industries Israel (Beit Haemek LTD). All cell lines were obtained through the NIH AIDS Research and Reference Reagent Program, Division of AIDS, NIAID, NIH. Jurkat E6-1 cells were from Dr. Arthur Weiss (Weiss A et al. 1984, J. Immunol. 133; 123-8), and Jurkat HXBc2 (4) cells expressing HIV-1 HXBc2 Rev and ENV proteins were from Dr. Joseph Sodroski (Cao, J. et al. 1996, J. Virol. 70; 1340-54). Cells were cultured every 3 to 4 days, and maintained in RPMI-1640 supplemented with the appropriate antibiotics at 37° C. with 5% CO₂ in a humidified incubator. For ENV expression, Jurkat HXBc2 (4) cells were transferred to medium without tetracycline three days prior to the experiments.
Peptide Synthesis, Fatty Acid Conjugation—Peptides were synthesized on Rink Amide MBHA resin by using the F-moc strategy as previously described (Merrifield, R. B. et al. 1982, Biochemistry, 21; 5020-31). C-terminally conjugated N-peptides contain a lysine residue at their C-terminus with an MTT side chain protecting group, enabling the conjugation of a fatty acid that required a special deprotection step under mild acidic conditions (2×1 min of 5% TFA in DCM and 30 min of 1% TFA in DCM). Conjugation of a fatty acid to the N-terminus was performed using standard F-moc chemistry. All peptides were cleaved from the resin by a TFA: DDW: TES (93.1:4.9:2 (v/v)) mixture, and purified by reverse phase high performance liquid chromatography (RP-HPLC) to >95% homogeneity. The molecular weight of the peptides was confirmed by platform LCA electrospray mass spectrometry.
Cell-Cell Fusion Inhibition Assay—The protocol utilizing Jurkat E6-1 and Jurkat HXBc2 cells for a cell-cell fusion assay was previously described (Huerta L. et al. 2002, Cytometry, 47; 100-6. In short, Jurkat E6-1 and Jurkat HXBc2 cells were labeled with DiI and DiD lypophilic fluorescent probes, respectively. The two cell populations were co-incubated for 6 h in a ratio of 1:1 in the presence of different concentrations of the inhibitory peptides. Cells that co-incubated without the presence of peptides served as an optimal fusion reference. Unlabeled cells that were handled similarly served as an intrinsic fluorescence control. Cells labeled separately with DiI or DiD were used to compensate for the optimal separation of fluorescent signals. Jurkat HXBc2 cells labeled with DiI were co-incubated with Jurkat HXBc2 cells labeled with DiD for a fusion background that was subtracted from the measurements of the experiment. The following alterations were applied to the original protocol: (i) 5 ul of a 1 mg/ml DiI or DiD solution in dimethylsulfoxide (DMSO) was added to 1 ml of 5×10⁶ cells/ml Jurkat E6-1 or Jurkat HXBc2 cells, respectively. (ii) Data from 150,000 events for each sample were collected on FACSort, upgraded to a FACSCalibur cell analyzer (Becton Dickinson), and further analyzed. Fitting of the data points was performed according to the equation derived from Hills' equation:

$$Y = B * ( \frac{{[ A ]}^{c}}{{X}^{c} + {[ A ]}^{c}} )$$

In this equation B is the maximum value, therefore it equals 100% fusion, A is the IC50 value, and c represents Hill's coefficient, in this particular case: the inhibitory oligomeric state of the peptide. For the fitting, we uploaded the X and Y values of the raw data (after subtracting the background) into a nonlinear least squares regression (curve fitter) program that provided the IC50 value (A of the equation), as well as the c value.
Virus Infectivity Assay—Fully infectious HIV-1 HXB2 concentrated virus stock was a kind gift of the AIDS Vaccine Program, SAIC. Experiments were done according to a P3 biological safety level. The infectivity of HIV-1 HXB2 was determined using the TZM-bl cell line as a reporter. Cells were added (2×10⁴ cells/well) to a 96-well clear-bottomed microtiter plate with 10% serum supplemented Dulbecco's modified eagle medium (DMEM). Plates were incubated at 37° C. for 18-24 hours to allow the cells to adhere. The media was then aspirated from each well and replaced with serum free DMEM containing 40 micrograms/mL DEAE-dextran. Stock dilutions of each peptide were prepared in DMSO so that each final concentration was achieved with 1% dilution. Upon addition of the peptides, the virus was added to the cells diluted in serum free DMEM containing 40 micrograms/mL DEAE-dextran. The plate was then incubated at 37° C. for 18 hours to allow the infection to occur. Luciferase activity was analyzed using the Steady-Glo Luciferase assay kit (Promega, Madison Wis.). All infectivity assays were performed in triplicate. IC50 Values were calculated from the fitted curve similarly to the cell-cell fusion assay.
Triple Staining Flow Cytometry Fusion Assay—For triple staining, the same cell-cell fusion inhibition assay experiment as described above was performed in the presence of NBD-labeled peptides. Cells labeled separately with DiI or DiD, and unlabeled cells in the presence of an NBD-labeled peptide were used to compensate for the optimal separation of the three fluorescent signals. For each data point 500,000 events were collected. The eight different possible combinations (triple, NBD, DiI, DiD, NBD+DiI, NBD+DiD, DiI+DiD, no label) were defined in the analysis software and the percentage of each one was calculated. The percentage of NBD labeling (peptide) on all cell types in relation to all available labeled cells in the system was calculated. This analysis provided us with the relative peptide concentration on cells.

$$\frac{N B D on cells}{All cells} = \frac{Triple + ( N B D + Dil ) + ( N B D + DiD ) \times 100}{Triple + DiD + Dil + ( DiD + Dil ) + ( N B D + DiD ) + ( N B D + Dil )}$$

Additionally, the percentage of NBD labeling (peptide) in cells labeled with DiD (effector) or DiI (target) cells was further calculated. Analysis of the data enabled us to assign a labeled peptide to different cell populations, namely, target or effector cells.

$$\frac{N B D on effector cells}{All effector cells} = \frac{Triple + ( N B D + DiD ) \times 100}{Triple + ( N B D + DiD ) + ( Dil + DiD ) + DiD}$$

$$\frac{N B D on target cells}{All target cells} = \frac{Triple + ( N B D + Dil ) \times 100}{Triple + ( N B D + Dil ) + ( Dil + DiD ) + Dil}$$

Circular Dichroism (CD) Spectroscopy—CD measurements were performed on an Aviv 202 spectropolarimeter. The spectra were scanned using a thermostatic quartz cuvette with a path length of 1 mm. Wavelength scans were performed at 25° C., the average recording time was 15 sec., in 1 nm steps, the wavelength range was 190-260 nm. Peptides were scanned at a concentration of 10 μM in 5 mM HEPES buffer and in a membrane mimetic environment of 1% LPC in ddH2O.

### Example 1

### Anchoring of N36 to the Membrane Increases its Inhibitory Activity

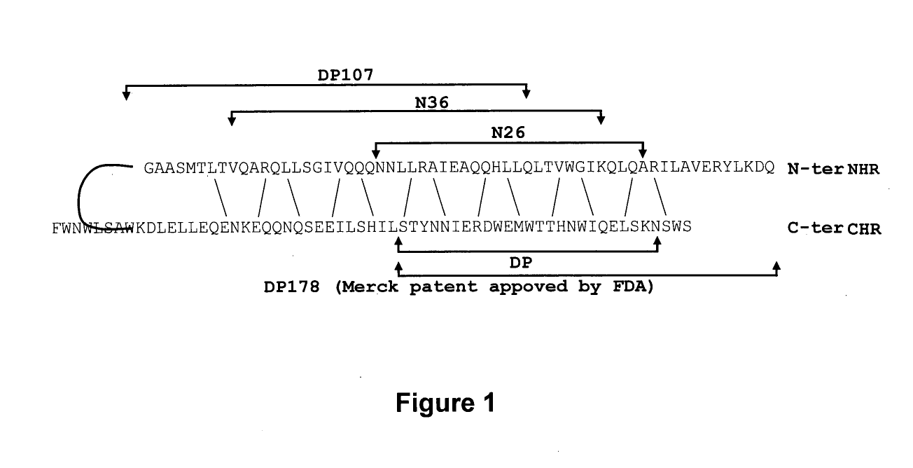

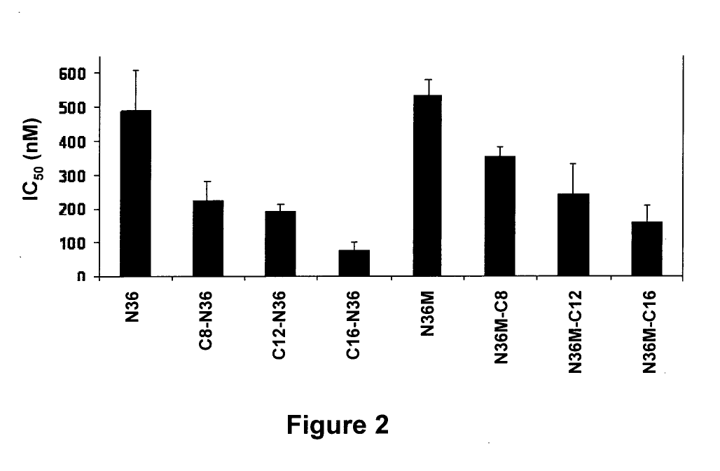

To scrutinize the effect of anchoring N36 to the membrane, we conjugated octanoic, dodecanoic, and palmitic acids to the N-terminus of N36 (Table 1). The resulting peptides C8-N36, C12-N36, and C16-N36 (Table 1) were examined in a cell-cell fusion inhibition assay and the results are shown in FIG. 1. A correlation was observed between the length of the conjugated fatty acid and the inhibitory activity of the N-conjugated N36 peptides. N36, C8-N36, C12-N36, and C16-N36 exhibited IC50 values of 488±119, 222±56, 190±21, and 72±27 nM, respectively. Interestingly, AcN36 was not active up to 2000 nM; therefore we refer to it as inactive. This correlates with previous studies demonstrating an IC₅₀ of 16000±2000 nM and 584±46 nM for the acetylated and non-acylated forms of N36, respectively (Bewley et al., 2002, J. Biol. Chem. 277:14238-45). Overall, our data reveal that the anchoring of N36 to the membrane significantly increases its inhibitory activity.

TABLE 1
Sequences, designations and IC50 values of the peptides and
their lipophilic conjugates in cell-cell fusion assay.

| Designation | Peptide sequence | IC50 nM |
|---|---|---|
| N36 | SGIVQQQNNLLRAIEAQQHLLQLTVWGIKQLQARIL | 488 ± 119 |
| AcN36 | Ac-SGIVQQQNNLLRAIEAQQHLLQLTVWGIKQLQARIL | Not active |
| C8-N36 | C8-SGIVQQQNNLLAIEAQQHLLQLTVWGIKQLQARIL | 222 ± 56 |
| C12-N36 | C12-SGIVQQQNNLLRAIEAQQHLLQLTVWGIKQLQARIL | 190 ± 21 |
| C16-N36 | C16-SGIVQQQNNLLRAIEAQQHLLQLTVWGIKQLQARIL | 72 ± 27 |
| N36M | SGIVQQQNNLLRAIEAQQHLLQLTVWGIKQLQARILK(δ-NH) | 531 ± 48 |
| N36M-C8 | SGIVQQQNNLLRAIEAQQHLLQLTVWGIKQLQARILK(δ-NH)-C8 | 354 ± 25 |
| N36M-C12 | SGIVQQQNNLLRAIEAQQHLLQLTVWGIKQLQARILK(δ-NH)-C12 | 241 ± 89 |
| N36M-C16 | SGIVQQQNNLLRAIEAQQHLLQLTVWGIKQLQARILK(δ-NH)-C16 | 159 ± 47 |

“Not Active” refers to a peptide or a peptide conjugate which IC₅₀ as determined by the cell-cell fusion assay was greater than 2 μM.

### Example 2

### The Orientation of Anchored N36 Towards the Endogenous CHR Region is not Crucial

To examine the importance of the proper orientation of the N36 peptide in relation to the pre-fusion conformation, we also conjugated octanoic, dodecanoic, and palmitic acids to the C-terminus of modified N36, termed N36M (Table 1). The parental peptide and the resulting fatty acid-conjugated peptides N36M, N36M-C8, N36M-C12, and N36M-C16 (Table 1) were examined in a cell-cell fusion inhibition assay and the results are presented in FIG. 1. Likewise, a correlation was observed between the length of the conjugated fatty acid and the inhibitory activity of the C-conjugated N36 peptides. N36M, N36M-C8, N36M-C12, and N36M-C16 exhibited IC₅₀ values of 531±48, 354±25, 241±89, and 159±47 nM, respectively. Since acetylating N36 abrogates its activity we added an acetyl group to N36M-C12 and N36M-C16 resulting in AcN36M-C12 and AcN36M-C16. Both lipopeptides were examined in a cell-cell fusion inhibition assay and exhibited IC50 values of 226±38, and 125±51 nM, respectively. Since these values are similar to those of N36M-C12 and N36M-C16 we can conclude without wishing to be bound by theory or mechanism of action, that the charge in their N-terminus does not influence their inhibitory ability, contrary to N36.

Interestingly, there was only a slight difference between the activities of N- and C-terminally conjugated peptides having the same fatty acid. This was in contrast with the results obtained with the C-helix peptide, in which there was a marked difference (˜30-fold) between them (Wexler-Cohen and Shai, 2007, Faseb J., 21:3677-84). Thus, without wishing to be bound by theory or mechanism of action, we can conclude that the length of the fatty acid is important, and it is correlated to the inhibitory activity, whereas primarily, the orientation of the peptides is not critical for their activity pattern.

### Example 3

### Inhibitory Curves Analysis Suggest a Different Mode of Inhibition for the Peptides of the Present Invention

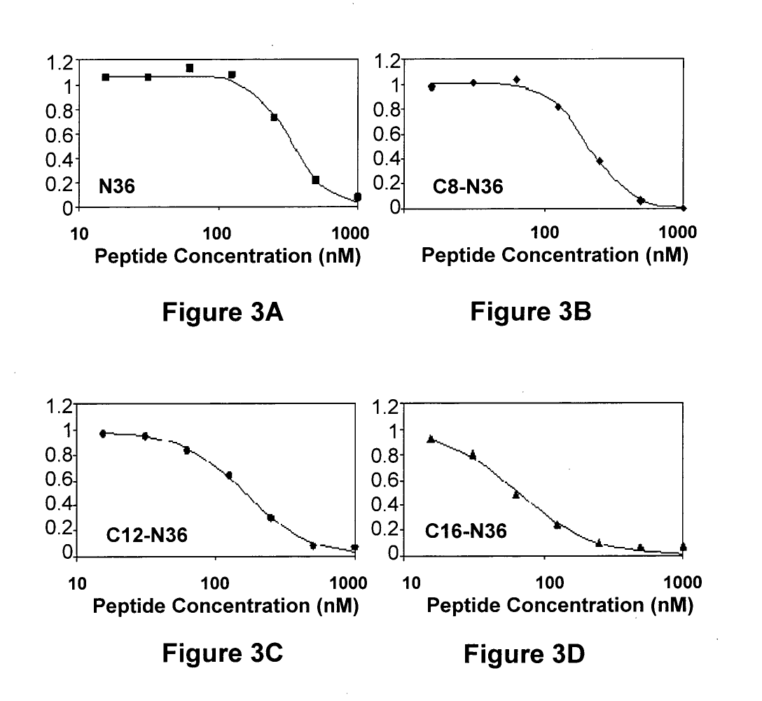

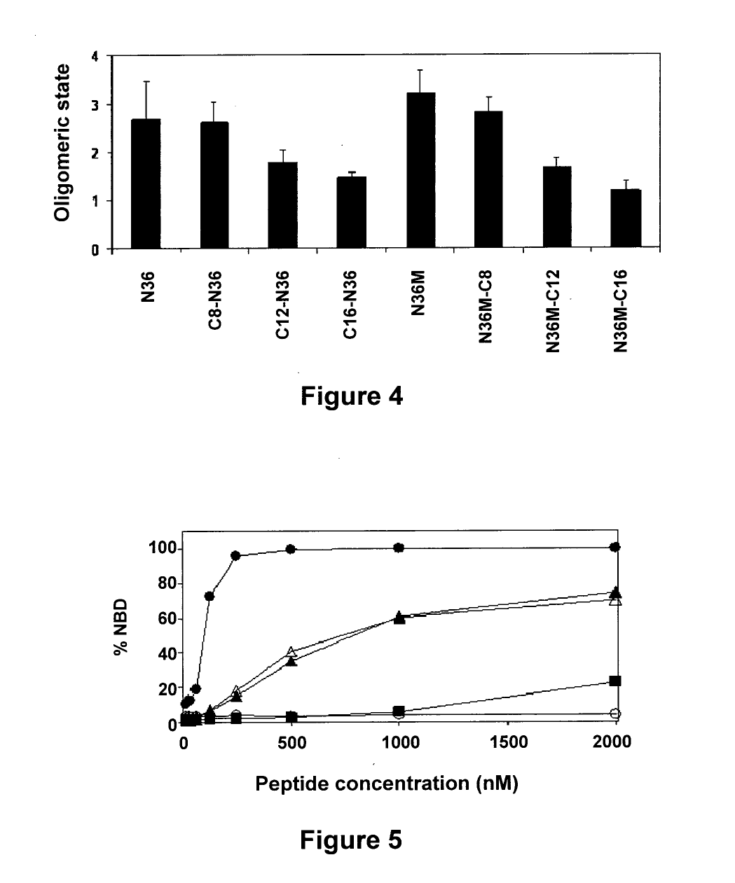

Representative experiments showing the inhibitory activity curves of N36 and its N-terminally fatty acid-conjugated analogs is presented in FIG. 3. FIG. 3 reveals different shapes of the binding curves for the different peptides shifted from sigmoid through a median shape to hyperbolic. A sigmoid shape can be explained by the tendency of N36 to oligomerize. Therefore, without wishing to be bound by theory or mechanism of action, we speculated that the different binding curves might be attributed to a different inhibitory oligomeric state of the peptides. Consequently, for optimal fitting, we employed an equation that contains a cooperativity parameter, indicative in this case, to the inhibitory oligomeric state of the peptide. Therefore, after a fit is achieved the c value represents the oligomeric state of the peptide. The values of the oligomerization parameters for the different peptides are presented in FIG. 4. The c values for the N-conjugated N36 peptides, namely: N36, C8-N36, C12-N36, and C16-N36 are 2.67, 2.61, 1.77, and 1.47 respectively. The c values for the C— conjugated N36 peptides, namely: N36M, N36M-C8, N36M-C12, and N36M-C16 are 3.19, 2.82, 1.67, and 1.19 respectively. These data reveal an interesting shift in the oligomerization tendency. It suggests that for the native peptides, N36, and N36M, the tendency is for the trimeric form. The longer the fatty acid the lower is the oligomerization value until it almost reaches a monomer with the C16-N36, and N36M-C16 peptides.

### Example 4

### Relative Concentration of the Peptides on the Membrane of the Cells

We tested whether the attachment of the fatty acids to the peptides allowed their anchoring to the cell membrane by utilizing a triple staining flow cytometry assay that incorporates fluorescently labeled target cells, effector cells, and inhibitory peptides (Wexler-Cohen and Shai, 2007, Faseb 1, 21:3677-84). This assay allowed the determination of the 1050 of the peptides, as well as the assignment of labeled peptides to cells. We analyzed the most and least active peptides (Table 2), namely, NBDN36 (parallel in its inhibitory activity to AcN36), NBDN36M-C16, and C16-N36NBD (FIG. 5). The NBDGCN4 peptide served as a negative control for a non-binding peptide, whereas, C16-NBDGCN4 served as a positive control for a strongly binding peptide. The data reveal a direct correlation between the activity of the N-helix peptides and their global concentration in the cells.

TABLE 2
Sequences and designations of the NBD labeled
peptides and their lipophilic conjugates.

| Designation | Peptide sequence |
|---|---|
| NBD^{N36} | NBD-NH-SGIVQQQNNLLRAIEAQQHLLQLTVWGIK |
| | QLQARIL |
| NBD^{N36M-C16} | NBD-NH-SGIVQQQNNLLRAIEAQQHLLQLTVWGIK |
| | QLQARILK(δ-NH)-C16 |
| C16-N36M_{NBD} | C16-NH-SGIVQQQNNLLRAIEAQQHLLQLTVWGIK |
| | QLQARILK(δ-NH)-NBD |
| NBD^{GCN4} | NBD-K(δ-NH)QIEDKIEEILSKIYHIENEIARIKK |
| | LIGER |
| C16-_{NBD}GCN4 | C16-NBD-K(δ-NH)QIEDKIEEILSKIYHIENEIA |
| | RIKKLIGER |

### Example 5

### Structure of the Peptides in Solution and in a Membrane Mimetic Environment Alone and in Combination with the C-Helix C34

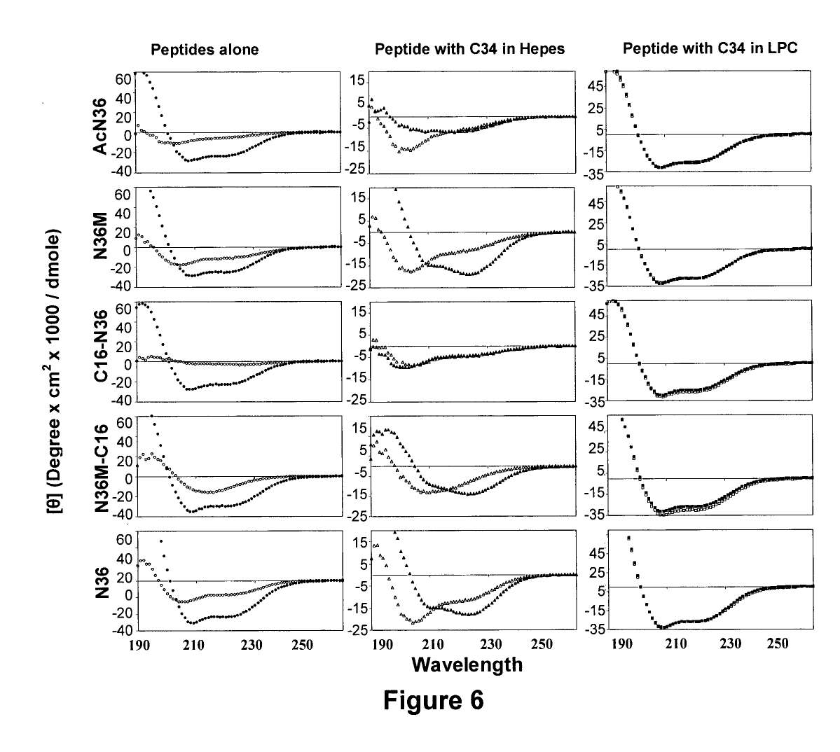

We determined the secondary structure of the most active and inactive peptides in solution to find out whether this feature correlates with their activity pattern. N36 and N36M exhibited α-helical structures in solution, whereas the structure of AcN36, C16-N36, and N36M-C16 was undefined (FIG. 6). The peptides' ability to create a core structure with C34 in solution was also monitored. The CD signal of each peptide was measured and their combined signal was calculated assuming that they do not interact with each other. This signal was compared to the actual signal monitored upon co-incubation of the two peptides together. If the couple interacts, we expect to see a difference between the two signals. AcN36 and C16-N36 were unable to create a core structure, whereas N36 and N36M-C16 did interact with C34 (FIG. 6) (Wexler-Cohen et al., J. Biol. Chem., 281:9005-10). The structure of the peptides alone, and their ability to create a core structure with C34 was also measured in a membrane mimetic environment (FIG. 6). Under these conditions, all the peptides exhibited α-helical structures. However, with all peptides, the non-interactive signal overlapped the experimental signal. Overall, these data demonstrate that the structure of the peptides and their ability or inability to create a core structure with C34 (in solution or in a membrane mimetic environment) cannot account for their activity pattern.

### Example 6

### Utilizing Known N36 Mutants to Explore the Inhibitory Mechanism

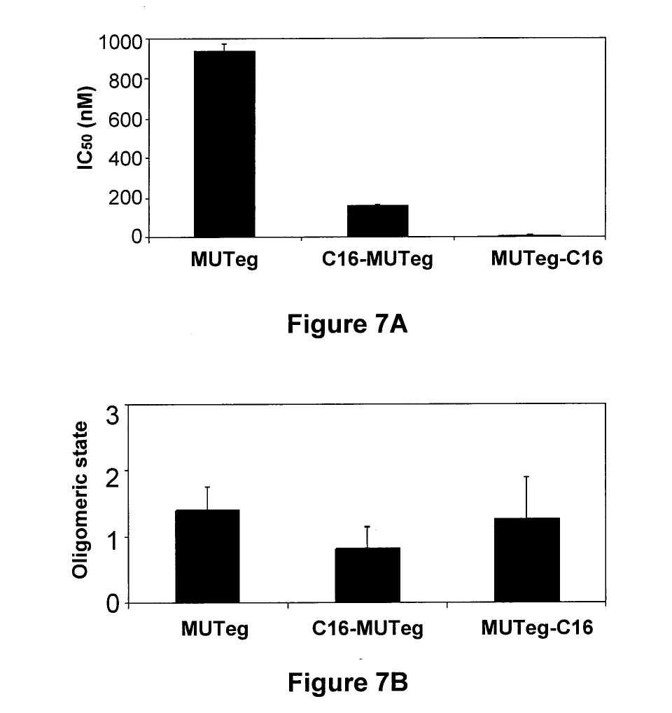

In order to investigate further the mechanism of inhibition we utilized known N36 mutants (Bewley et al., 2002, J. Biol. Chem., 277:14238-45). The first was N36 MUTe,g which contains mutations in its e and g positions. These mutations preserve its ability to self-assemble into trimers, but it cannot interact with the CHR. The second mutant was N36 MUTa,d which contains mutations in its a and d positions knocking out its ability to interact with itself, thus leading to inability to create the internal coiled-coil (Table 3). These mutants demonstrated that the NHR can inhibit by preventing the formation of the viral NHR coil-coiled (probably as a monomer or dimer), or by binding to the CHR domain to prevent six helix bundle formation (probably as a trimer) (Bewley et al., 2002, J. Biol. Chem., 277:14238-45). We conjugated a palmitic acid to the N or C-terminus of both of them and determined their IC50 inhibitory values. The N36 MUTa,d was inactive alone and when conjugated to palmitic acid, without wishing to be bound by theory or mechanism of action we speculate it is because it could not bind itself, as well as the CHR domain, therefore both modes of inhibitions could not take place. Strikingly however, the attachment of palmitic acid to N36 MUTe,g caused an increase of 7-fold to 100-fold in its IC50 compared to the soluble peptide, depending on the directionality of the conjugation. N36 MUTe,g, C16-N36 MUTe,g, and N36 MUTe,g-C16 exhibited IC50 values of 936±36, 162±4, and 8.8±4 nM, respectively (FIG. 7A). Such preference was not observed with the wild type N36 which preserve binding to the CHR region. Furthermore, without wishing to be bound by theory or mechanism of action, whereas data analysis suggest a trimeric and monomeric modes of inhibition for the wild type N36 and its palmitic acid conjugates, respectively (FIG. 7B), a mainly monomeric mode of inhibition for both the soluble N36 MUTe,g, and its palmitoylated forms is suggested.

TABLE 3
Sequences and designations of the N36 mutated peptides and
their lipophilic conjugates.

| Designation | Peptide sequence | IC50 nM |
|---|---|---|
| N36 MUTe,g | SGIDQEQNNLTRLIEAQIHELQLTQWKIKQLLARILK(δ-NH) | 936 ± 36 |
| C16-N36 MUTe,g | SGIDQEQNNLTRLIEAQIHELQLTQWKIKQLLARILK(δ-NH) | 162 ± 4 |
| N36 MUTe,g-C16 | SGIDQEQNNLTRLIEAQIHELQLTQWKIKQLLARILK(δ-NH) | 8.8 ± 4 |
| N36 MUTa,d | SGIVQQLNNQLRAEEANQHLEQLSVWGSKQNQARRLK(δ-NH) | Not active |
| C16-N36 MUTa,d | SGIVQQLNNQLRAEEANQHLEQLSVWGSKQNQARRLK(δ-NH) | Not active |
| N36 MUTa,d-C16 | SGIVQQLNNQLRAEEANQHLEQLSVWGSKQNQARRLK(δ-NH) | Not active |

“Not Active” refers to a peptide or a peptide conjugate which IC₅₀ as determined by the cell-cell fusion assay was greater than 2 μM.

### Example 7

### The Relative Concentration of the Peptides in Specific Cell Populations

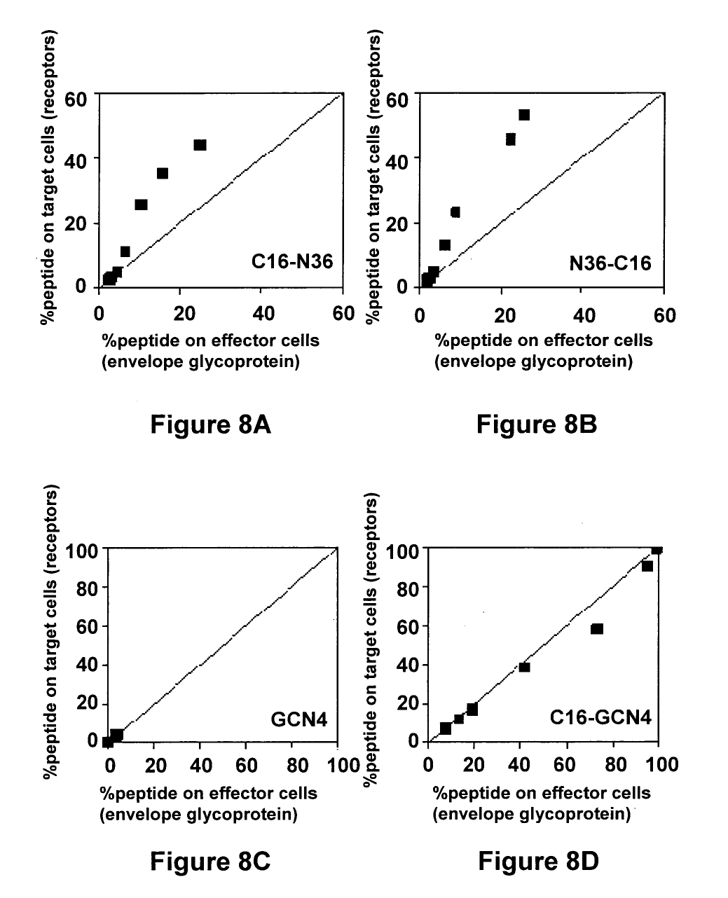

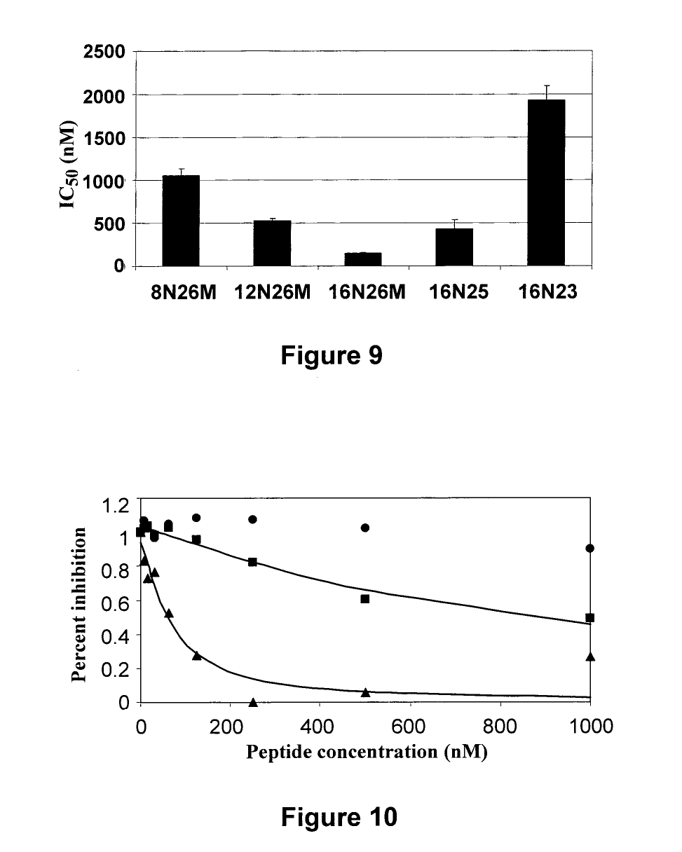

To examine whether the peptides have an enhanced tendency to bind the cells with the receptors (target cells), or those with the ENV glycoprotein (effector cells), in a dynamic fusion process, we employed a triple staining assay. Fluorescently labeled peptides were incubated with differently labeled effector and target cells, exactly according to the protocol of the cell-cell fusion assay. The fusion was allowed to take place and then the sample was washed and measured by FACS. Further analysis, enabled us to compare the relative level of the peptide's binding for each cell population (FIG. 8). The NBDGCN4 peptide served as a negative control for a non-binding peptide, whereas C16-NBDGCN4 served as a positive control for a strongly binding peptide without preference for a specific cell population. A line is drawn in each panel to emphasize where we would expect the data in case there is no preference among the different populations. Since the NBDGCN4 peptide does not bind the membranes, all the data points are concentrated in the lower left-hand corner. We can conclude that (under the same conditions as used for the experiments determining the inhibitory activity of the peptides) there is a tendency of the conjugated N36 peptides to reside more on target than on effector cells.

### Example 8

### Inhibitory Activity of the Short Peptides of the Present Invention

We synthesized an N-peptide, named N26, that is shifted in its amino acid sequence in regard to the known in the art N36 peptide (Yingying Le et al. 2000, Clin. Immun. 96; 236-42); The peptide of the present invention starts four amino acids upstream from the N-terminus of N36 and ends 14 amino acids upstream from the C-terminus of N36 (FIG. 1). The C-terminal sequence of N36, which was deleted from the peptides of the present invention, comprises the pocket of the six-helix bundle structure. Conjugation of hydrophobic moieties such as fatty acids with increasing lengths, cholesterol or vitamin E to the N-terminus of the peptide of the present invention resulted in increased inhibitory activity as demonstrated by the IC₅₀ values (using the cell-cell fusion assay): 1075±90, 473±74, 148±4, 150±20 and 400±60 nM for C8-N26M, C12-N26M, C16-N26M, Cholesterol-N26M and VitE-N26M respectively (Tables 4 and 5). The inhibitory activity of the peptides and peptide conjugates according to some embodiments of the present invention was further demonstrated using the virus-cell fusion assay which resulted with IC50 values of 338±16, 293±27, 182±15, 10±1 nM, 25±1 nM and 50±1 nM for N26M, C8-N26M, C12-N26M, C16-N26M, Cholesterol-N26M and VitaminE-N26M respectively (Table 5). Conjugation of fatty acids to the C-terminus of the peptide resulted in inactive peptides. Shortening of the peptide by 2-4 amino acids reduced the inhibitory activity of the conjugated peptide; Truncation of two C-terminally amino acids resulted in IC₅₀ value of 484±60 nM and 30±5 nM for C16-N25 as determined by the cell-cell fusion assay and virus-cell fusion assay respectively, and truncation of additional two C-terminus amino acids resulted in IC₅₀ value of 1931±187 nM and 100±24 nM for C16-N23 as determined by the cell-cell fusion assay and virus-cell fusion assay respectively. By comparing the inhibitory activity of the lipopeptides of the present invention with that of N36 peptide in the same system (IC₅₀ value of 488±119 nM (IC50 value determined by cell-cell fusion assay), we can conclude that the lipopeptides of the present invention display enhanced inhibitory ability.

TABLE 4
Sequences and designations of the peptides of the present invention
and lipophilic conjugates thereof.

| | | |
|---|---|---|
| | | IC₅₀ (nM) |
| | | In cell-cell |
| Designation | Peptide sequence | fusion assay |

A

| | | |
|---|---|---|
| N26 | RQLLSGIVQQQNNLLRAIEAQQHLLQ | Not active |
| C12-N26 | C12-RQLLSGIVQQQNNLLRAIEAQQHLLQ | 814 |
| C16-N26 | C16-RQLLSGIVQQQNNLLRAIEAQQHLLQ | 66 |

B

| | | |
|---|---|---|
| N26M | RQLLSGIVQQQNNLLRAIEAQQHLLQK | Not active |
| C8-N26M | C8-RQLLSGIVQQQNNLLRAIEAQQHLLQK | 1075 ± 90 |
| C12-N26M | C12-RQLLSGIVQQQNNLLRAIEAQQHLLQK | 473 ± 74 |
| C16-N26M | C16-RQLLSGIVQQQNNLLRAIEAQQHLLQK | 148 ± 4 |
| Cholesterol-N26M | Chol-RQLLSGIVQQQNNLLRAIEAQQHLLQK | 150 ± 20 |
| VitE-N26M | VitE-RQLLSGIVQQQNNLLRAIEAQQHLLQK | 400 ± 60 |
| N26M-C8 | RQLLSGIVQQQNNLLRAIEAQQHLLQK-C8 | Not active |
| N26M-C12 | RQLLSGIVQQQNNLLRAIEAQQHLLQK-C12 | Not active |
| N26M-C16 | RQLLSGIVQQQNNLLRAIEAQQHLLQK-C16 | Not active |

C

| | | |
|---|---|---|
| N25 | RQLLSGIVQQQNNLLRAIEAQQHLL | Not active |
| C16-N25 | C16-RQLLSGIVQQQNNLLRAIEAQQHLL | 484 ± 60 |

D

| | | |
|---|---|---|
| N23 | RQLLSGIVQQQNNLLRAIEAQQH | Not active |
| C16-N23 | C16-RQLLSGIVQQQNNLLRAIEAQQH | 1931 ± 187 |

E

| | | |
|---|---|---|
| N22 | SGIVQQQNNLLRAIEAQQHLLQ | Not active |
| C8-N22 | C8-SGIVQQQNNLLRAIEAQQHLLQ | Not active |
| C12-N22 | C12-SGIVQQQNNLLRAIEAQQHLLQ | Not active |

F

| | | |
|---|---|---|
| Sh-Mut e,g | HQTLSGIDQEQNNLTRLIEAQIHELQ | Not active |
| C16-Sh-Mut e,g | C16-HQTLSGIDQEQNNLTRLIEAQIHELQ | Not active |
| Sh-Mut e,g-C16 | HQTLSGIDQEQNNLTRLIEAQIHELQ-C16 | Not active |
| Sh-RQLL-Mut e,g | RQLLSGIDQEQNNLTRLIEAQIHELQ | Not active |
| C16-Sh-RQLL-Mut e,g | C16-RQLLSGIDQEQNNLTRLIEAQIHELQ | Not active |
| Sh-RQLL-Mut e,g-C16 | RQLLSGIDQEQNNLTRLIEAQIHELQ-C16 | Not active |

“Not Active” refers to a peptide or a peptide conjugate which IC₅₀ determined by the cell-cell fusion assay was greater than 2 μM.

TABLE 5
Inhibition concentrations of lipophilic conjugates of the present
invention as determined by virus-cell fusion assay.

| | | IC₅₀ (nM) |
|---|---|---|
| | | In virus-cell |
| Designation | Peptide sequence | fusion assay |
| N26M | RQLLSGIVQQQNNLLRAIEAQQHLLQK | 338 ± 16 |
| C8-N26M | C8-RQLLSGIVQQQNNLLRAIEAQQHLLQK | 293 ± 27 |
| C12-N26M | C12-RQLLSGIVQQQNNLLRAIEAQQHLLQK | 182 ± 15 |
| C16-N26M | C16-RQLLSGIVQQQNNLLRAIEAQQHLLQK | 10 ± 1 |
| Cholesterol-N26M | Chol-RQLLSGIVQQQNNLLRAIEAQQHLLQK | 25 ± 1 |
| VitE-N26M | VitE-RQLLSGIVQQQNNLLRAIEAQQHLLQK | 50 ± 1 |
| N25 | RQLLSGIVQQQNNLLRAIEAQQHLL | Not active |
| C16-N25 | C16-RQLLSGIVQQQNNLLRAIEAQQHLL | 30 ± 5 |
| N23 | RQLLSGIVQQQNNLLRAIEAQQH | Not active |
| C16-N23 | C16-RQLLSGIVQQQNNLLRAIEAQQH | 100 ± 24 |
| Sh-Mut e,g | HQTLSGIDQEQNNLTRLIEAQIHELQ | Not active |
| C16-Sh-Mut | C16-HQTLSGIDQEQNNLTRLIEAQIHELQ | 643 ± 137 |
| e,g | | |
| Sh-RQLL-Mut | RQLLSGIDQEQNNLTRLIEAQIHELQ-C16 | 870 ± 120 |
| e,g-C16 | | |

“Not Active” refers to a peptide or a peptide conjugate which IC₅₀ determined by the virus-cell fusion assay was greater than 1 μM.

1-56. (canceled)

57. A lipophilic conjugate comprising an isolated peptide coupled to a hydrophobic moiety, the peptide comprising the sequence of the formula (I):
(I)
X₁-X₂-X₃-X₄-Ser-Gly-Ile-X₅-Gln-X₆-Gln-Asn-Asn-Leu-
X₇-Arg-X₈-Ile-Glu-Ala-Gln-X₉-His
wherein;
X₁ is selected from the group consisting of an arginine and a lysine amino acid residue;
X₂ is selected from the group consisting of: arginine, lysine, glutamine and asparagine amino acid residues;
X₃ and X₄ are each independently selected from the group consisting of: leucine, isoleucine, valine and methionine amino acid residues;
X₅ is selected from the group consisting of a valine, a leucine, an isoleucine, an aspartic acid and a glutamic acid amino acid residue;
X₆ is selected from the group consisting of a glutamine, an asparagine, a glutamic acid and an aspartic acid amino acid residue;
X₇ is selected from the group consisting of a threonine, a serine, a leucine, an isoleucine and a valine amino acid residue;
X₈ is selected from the group consisting of a leucine, an isoleucine, a valine and an alanine amino acid residue;
X₉ is selected from the group consisting of an isoleucine, a leucine, a valine, a glutamine and an asparagine, amino acid residue;
wherein the hydrophobic moiety is conjugated to the N-terminus or C-terminus of said isolated peptide;
and wherein the lipophilic conjugate is capable of inhibiting protein-induced membrane fusion.

58. The lipophilic conjugate according to claim 57, wherein the hydrophobic moiety is conjugated to the N-terminus of the isolated peptide.

59. The lipophilic conjugate according to claim 57, wherein the hydrophobic moiety comprises an aliphatic group comprising at least 6 carbon atoms, or wherein the hydrophobic moiety is a fatty acid or a sterol or a fat soluble vitamin selected from the group consisting of vitamin A, vitamin D, vitamin E and vitamin K.

60. The lipophilic conjugate according to claim 59, wherein the fatty acid is selected from saturated, unsaturated, monounsaturated and polyunsaturated fatty acids, or wherein the fatty acid consists of at least six carbon atoms.

61. The lipophilic conjugate according to claim 60, wherein the fatty acid is selected from the group consisting of decanoic acid, undecanoic acid, dodecanoic acid, myristic acid, palmitic acid, stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, and cerebronic acid.

62. The lipophilic conjugate according to claim 57, wherein the isolated peptide comprises up to 30 amino acid residues, or wherein the isolated peptide further comprises at least one positively charged amino acid residue at the C-terminus, N-terminus or both.

63. The lipophilic conjugate according to claim 57, wherein the protein inducing membrane fusion is an envelope surface glycoprotein selected from envelope surface glycoproteins of HIV and simian immunodeficiency virus.

64. The lipophilic conjugate according to claim 63, wherein the envelope surface glycoprotein of HIV is HIV-1 gp41.

65. The lipophilic conjugate according to claim 62, wherein the at least one positively charged amino acid residue is added to the C-terminus.

66. The lipophilic conjugate according to claim 57, wherein X₁ is an arginine, X₂ is a glutamine, X₃ is a leucine and X₄ is a leucine.

67. The lipophilic conjugate according to claim 57, wherein the sequence of the isolated peptide is as set forth in any one of SEQ ID NOS: 2, 4, 6, 8, 13, 15, 17 and 19.

68. The lipophilic conjugate according to claim 57, wherein the sequence of the isolated peptide is as set forth in any one of SEQ ID NOS: 3, 5, 7, 9, 14, 16, 18 and 20.

69. A pharmaceutical composition comprising as an active ingredient a lipophilic conjugate according to claim 57 and a pharmaceutically acceptable carrier or diluent.

70. A method for inhibiting membrane protein assembly in a cell comprising contacting the cell with an effective amount of a lipophilic conjugate according to claim 57, thereby inhibiting the membrane protein assembly.

71. A method for inhibiting infection of a cell by a virus comprising contacting the cell with an effective amount of a lipophilic conjugate according to claim 57, thereby inhibiting the infection of the cell.

72. A method for inhibiting virus replication or transmission in a subject comprising administering to the subject in need of such treatment a therapeutically effective amount of a pharmaceutical composition according to claim 69, thereby inhibiting the virus replication or transmission.

73. A lipophilic conjugate comprising an isolated peptide coupled to a hydrophobic moiety, the isolated peptide comprising the sequence of formula (II):
(II)
Ser-Gly-Ile-X₁-Gln-X₂-Gln-Asn-Asn-Leu-X₃-Arg-X₄-
Ile-Glu-Ala-Gln-X₅-His-X₆-Leu-Gln-Leu-Thr-X₇-Trp-
X₈-Ile-Lys-Gln-Leu-X₉-Ala-Arg-Ile-Leu
wherein:
X₁ is selected from the group consisting of aspartic acid, a glutamic acid, a valine, a leucine and an isoleucine amino acid residue;
X₂ is selected from the group consisting of an aspartic acid, a glutamic acid, an asparagine and a glutamine amino acid residue;
X₃ is selected from the group consisting of a threonine, a serine, a leucine, an isoleucine and a valine amino acid residue;
X₄ is selected from the group consisting of a leucine, an isoleucine, a valine and an alanine amino acid residue;
X₅ is selected from the group consisting of a leucine, an isoleucine, a valine, a glutamine and an asparagine, amino acid residue;
X₆ is selected from the group consisting of a leucine, an isoleucine, a valine, an aspartic acid and a glutamic acid;
X₇ is selected from the group consisting of a glutamine, an asparagine, a leucine, an isoleucine and a valine amino acid residue;
X₈ is selected from the group consisting of a lysine, an arginine and a glycine amino acid residue;
X₉ is selected from the group consisting of a leucine, an isoleucine, a valine, a glutamine or an asparagine, amino acid residue;
wherein said hydrophobic moiety is conjugated to the N-terminus, C-terminus or both termini of said isolated peptide, and wherein said lipophilic conjugate is capable of inhibiting protein-induced membrane fusion.

74. The lipophilic conjugate according to claim 73, wherein the amino acid sequence of the isolated peptide is as set forth in SEQ ID NO: 11 or SEQ ID NO: 12.

75. The lipophilic conjugates according to claim 73, wherein said isolated peptide further comprising at least one positively charged amino acid residue at the carboxy terminus, amino terminus or both, or wherein the isolated peptide comprises up to 40 amino acid residues.

76. The lipophilic conjugate according to claim 73, wherein the hydrophobic moiety comprises an aliphatic group comprising at least six carbon atoms, or wherein the hydrophobic moiety is a fatty acid or a sterol or a fat soluble vitamin selected from the group consisting of vitamin A, vitamin D, vitamin E and vitamin K.

77. The lipophilic conjugate according to claim 76, wherein the fatty acid is selected from saturated, unsaturated, monounsaturated and polyunsaturated fatty acids, or wherein the fatty acid consists of at least six carbon atoms.

78. The lipophilic conjugate according to claim 76, wherein the fatty acid is selected from the group consisting of decanoic acid, undecanoic acid, dodecanoic acid, myristic acid, palmitic acid, stearic acid, arachidic acid, lignoceric acid, palmitoleic acid, oleic acid, linoleic acid, linolenic acid, arachidonic acid, trans-hexadecanoic acid, elaidic acid, lactobacillic acid, tuberculostearic acid, and cerebronic acid.

79. The lipophilic conjugate according to claim 73, wherein the protein inducing membrane fusion is an envelope surface glycoprotein selected from envelope surface glycoproteins of HIV and simian immunodeficiency virus.

80. The lipophilic conjugate according to claim 79, wherein the envelope surface glycoprotein of HIV is HIV-1 gp41.

81. A pharmaceutical composition comprising as an active ingredient a lipophilic conjugate according to claim 73 and a pharmaceutically acceptable carrier or diluent.

82. A method for inhibiting membrane protein assembly in a cell comprising contacting the cell with an effective amount of a lipophilic conjugate according to claim 73, thereby inhibiting the membrane protein assembly.

83. A method for inhibiting infection of a cell by a virus comprising contacting the cell with an effective amount of a lipophilic conjugate according to claim 73, thereby inhibiting the infection of the cell.

84. A method for inhibiting virus replication or transmission in a subject comprising administering to the subject in need of such treatment a therapeutically effective amount of a pharmaceutical composition according to claim 81, thereby inhibiting the virus replication or transmission.


SEQUENCE LISTING (22 sequences)

SEQ ID NO: 1 - protein, 23 aa, Artificial Sequence
  Synthetic peptide
XXXXSGIXQXQNNLXRXIEAQXH

SEQ ID NO: 2 - protein, 23 aa, Artificial Sequence
  Synthetic peptide
RQLLSGIVQQQNNLLRAIEAQQH

SEQ ID NO: 3 - protein, 23 aa, Artificial Sequence
  Synthetic peptide
RQLLSGIDQEQNNLTRLIEAQIH

SEQ ID NO: 4 - protein, 24 aa, Artificial Sequence
  Synthetic peptide
RQLLSGIVQQQNNLLRAIEAQQHL

SEQ ID NO: 5 - protein, 24 aa, Artificial Sequence
  Synthetic peptide
RQLLSGIDQEQNNLTRLIEAQIHE

SEQ ID NO: 6 - protein, 25 aa, Artificial Sequence
  Synthetic peptide
RQLLSGIVQQQNNLLRAIEAQQHLL

SEQ ID NO: 7 - protein, 25 aa, Artificial Sequence
  Synthetic peptide
RQLLSGIDQEQNNLTRLIEAQIHEL

SEQ ID NO: 8 - protein, 26 aa, Artificial Sequence
  Synthetic peptide
RQLLSGIVQQQNNLLRAIEAQQHLLQ

SEQ ID NO: 9 - protein, 26 aa, Artificial Sequence
  Synthetic peptide
RQLLSGIDQEQNNLTRLIEAQIHELQ

SEQ ID NO: 10 - protein, 36 aa, Artificial Sequence
  Synthetic peptide
SGIXQXQNNLXNXIEAQXHXLQLTXWXIKQLXARIL

SEQ ID NO: 11 - protein, 36 aa, Artificial Sequence
  Synthetic peptide
SGIVQQQNNLLRAIEAQQHLLQLTVWGIKQLQARIL

SEQ ID NO: 12 - protein, 36 aa, Artificial Sequence
  Synthetic peptide
SGIDQEQNNLTRLIEAQIHELQLTQWKIKQLLARIL

SEQ ID NO: 13 - protein, 24 aa, Artificial Sequence
  Synthetic peptide
RQLLSGIVQQQNNLLRAIEAQQHK

SEQ ID NO: 14 - protein, 24 aa, Artificial Sequence
  Synthetic peptide
RQLLSGIDQEQNNLTRLIEAQIHK

SEQ ID NO: 15 - protein, 25 aa, Artificial Sequence
  Synthetic peptide
RQLLSGIVQQQNNLLRAIEAQQHLK

SEQ ID NO: 16 - protein, 25 aa, Artificial Sequence
  Synthetic peptide
RQLLSGIDQEQNNLTRLIEAQIHEK

SEQ ID NO: 17 - protein, 26 aa, Artificial Sequence
  Synthetic peptide
RQLLSGIVQQQNNLLRAIEAQQHLLK

SEQ ID NO: 18 - protein, 26 aa, Artificial Sequence
  Synthetic peptide
RQLLSGIDQEQNNLTRLIEAQIHELK

SEQ ID NO: 19 - protein, 27 aa, Artificial Sequence
  Synthetic peptide
RQLLSGIVQQQNNLLRAIEAQQHLLQK

SEQ ID NO: 20 - protein, 27 aa, Artificial Sequence
  Synthetic peptide
RQLLSGIDQEQNNLTRLIEAQIHELQK

SEQ ID NO: 21 - protein, 37 aa, Artificial Sequence
  Synthetic peptide
SGIVQQQNNLLRAIEAQQHLLQLTVWGIKQLQARILK

SEQ ID NO: 22 - protein, 37 aa, Artificial Sequence
  Synthetic peptide
SGIDQEQNNLTRLIEAQIHELQLTQWKIKQLLARILK